(19) United States
(12) Patent Application Publication
(10) Pub. No.: US 2026/0265370 A1
(43) Pub. Date: Sep. 10, 2026

(54) COMBINATION THERAPIES INVOLVING CLAUDIN 18.2 ANTAGONISTS FOR TREATMENT OF CANCER

(71) Applicants: SUZHOU TRANSCENTA THERAPEUTICS CO., LTD., Suzhou, Jiangsu (CN); TRANSCENTA HOLDING LIMITED, Grand Cayman (KY); TRANSCENTA THERAPEUTICS, INC., Princeton, NJ (US)

(72) Inventors: Xueming QIAN, Suzhou, Jiangsu (CN); Caroline GERMA, Suzhou, Jiangsu (CN); Chuan QI, Suzhou, Jiangsu (CN); Li XU, Suzhou, Jiangsu (CN)

(21) Appl. No.: 19/489,267

(22) PCT Filed: May 31, 2024
(86) PCT No.: PCT/CN2024/096805

§ 371 Date: Dec. 2, 2025

(30) Foreign Application Priority Data
Jun. 3, 2023 (WO) PCT/CN2023/098182
May 16, 2024 (WO) PCT/CN2024/093779

(51) Int. Cl. C07K 16/28; A61K 9/00; A61K 39/00; A61K 45/06; A61K 47/68; A61P 35/00; G01N 33/575

(52) U.S. Cl. CPC C07K 16/28; A61K 9/0019; A61K 45/06; A61K 47/6849; A61P 35/00; C07K 16/2818; C07K 16/2827; G01N 33/5759; A61K 2039/505; A61K 2039/542; A61K 2039/543; A61K 2039/545; C07K 2317/24; C07K 2317/31; C07K 2317/52; C07K 2317/565; C07K 2317/732; C07K 2317/734; C07K 2317/76; G01N 2333/70532; G01N 2474/20

(57) ABSTRACT

Combination therapies of a CLDN18.2 antagonist, chemotherapy and optionally immunotherapy for treating CLDN18.2-expressing cancers.

### CROSS-REFERENCE TO RELATED APPLICATIONS

This application claims priority to International Application No. PCT/CN2023/098182, filed on Jun. 3, 2023, and to International Application No. PCT/CN2024/093779, filed on May 16, 2024, the disclosure of each of which is incorporated herein by reference in its entirety.

### SEQUENCE LISTING

The sequence listing that is contained in the file named “063694-8016WO03_ST26”, which is 50,877 bytes and was created on May 28, 2024, is filed herewith by electronic submission and is incorporated by reference herein.

### TECHNICAL FIELD

The present disclosure generally relates to cancer therapy involving a claudin 18.2 (CLDN18.2) antagonist in combination with a second therapy (e.g., chemotherapy and/or immunotherapy) for treating a cancer expressing Claudin 18.2.

### BACKGROUND OF THE INVENTION

The cell surface antigen claudin-18 spice variants 2 (CLDN18.2) is considered as an ideal target for the development of therapeutics for the treatment of cancers. In normal tissues, it is confined to differentiated epithelial cells of gastric mucosa, and absent from the gastric stem cell zone and other normal tissues. However, aberrant ectopic expression of CLDN18.2 has been reported in gastric, pancreatic, ovarian, biliary and lung adenocarcinomas (Sahin U et al., Claudin-18 splice variant 2 is a pan-cancer target suitable for therapeutic antibody development, Clin Cancer Res. 2008 Dec. 1; 14(23):7624-34.) and also confirmed by other similar studies (Karanjawala Z E et al., New markers of pancreatic cancer identified through differential gene expression analyses: claudin 18 and annexin A8. Am J Surg Pathol. 2008 February; 32(2):188-96.; Micke P et al., Aberrantly activated claudin 6 and 18.2 as potential therapy targets in non-small-cell lung cancer. Int J Cancer. 2014 Nov. 1; 135(9):2206-14.; Keira Y et al., An immunohistochemical marker panel including claudin-18, maspin, and p53 improves diagnostic accuracy of bile duct neoplasms in surgical and presurgical biopsy specimens. Virchows Arch. 2015 March; 466(3):265-77.). Current available information indicates that CLDN18.2 is a promising therapeutic target for the treatment of solid tumors.

Current available CLDN18.2-directed therapies have shown therapeutic efficacies in certain patients with CLDN18.2-expressing cancers, but the efficacy benefits limited to those patients having high CLDN18.2-expression. However, there are still a large proportion of patients having medium or low CLDN18.2-expressing cancers whose medical needs have been unmet.

Therefore, significant need exists for novel CLDN18.2-directed therapies for these larger proportion of patients.

### SUMMARY OF THE INVENTION

The present disclosure provides, among others, a method for treating a method of identifying and treating a subject having a Claudin 18.2 (CLDN18.2)-expressing cancer who may benefit from a treatment comprising a CLDN18.2 antagonist in combination with chemotherapy, the method comprising: determining the level of CLDN18.2 protein in a cancer sample (e.g., in a tumor tissue sample), and selecting a subject having medium-to-high (e.g. medium or high), or low level of the CLDN18.2 protein for the treatment.

In another aspect, the present disclosure provides a method for treating a CLDN18.2-expressing cancer in a human subject in need thereof, comprising: administering to the subject a therapeutically effective amount of a CLDN18.2 antagonist in combination with chemotherapy, wherein the subject is determined to have medium-to-high (e.g. medium or high), or low level of CLDN18.2 expression in a cancer sample (e.g., in a tumor tissue sample).

In some embodiments, the method described herein wherein: a) the high level of CLDN18.2 protein or CLDN18.2 expression corresponds to at least 70% of the cancer cells in the cancer sample (e.g., tumor tissue sample) having an expression level of CLDN18.2 characterized by membrane staining intensity of at least 2+ as measured using an anti-CLDN18.2 diagnostic antibody on Leica Bond III LDT platform or an equivalent readout as measured on another test platform; b) the medium level of CLDN18.2 protein or CLDN18.2 expression corresponds to at least 40% of the cancer cells in the cancer sample (e.g., tumor tissue sample) having an expression level of CLDN18.2 characterized by membrane staining intensity of at least 2+, and less than 70% of the cancer cells in the cancer sample (e.g., tumor tissue sample) having an expression level of CLDN18.2 characterized by membrane staining intensity of at least 2+ as measured using an anti-CLDN18.2 diagnostic antibody on Leica Bond III LDT platform or an equivalent readout as measured on another test platform; and c) the low level of CLDN18.2 protein or CLDN18.2 expression corresponds to at least 1-10% (e.g., at least 1%, at least 5% or at least 10%) of the cancer cells in the cancer sample (e.g., tumor tissue sample) having an expression level of CLDN18.2 characterized by membrane staining intensity of at least 1+, and less than 40% of the cancer cells in the cancer sample (e.g., tumor tissue sample) having an expression level of CLDN18.2 characterized by membrane staining intensity of at least 2+ or 3+ as measured using an anti-CLDN18.2 diagnostic antibody on Leica Bond III LDT platform or an equivalent readout as measured on another test platform.

In some embodiments, the method described herein wherein: a) the high level of CLDN18.2 protein or CLDN18.2 expression corresponds to at least 70% of the cancer cells in the cancer sample (e.g., tumor tissue sample) having an expression level of CLDN18.2 characterized by membrane staining intensity of at least 2+ as measured using 14G11 on Leica Bond III LDT platform or an equivalent readout as measured on another test platform; b) the medium level of CLDN18.2 protein or CLDN18.2 expression corresponds to at least 40% of the cancer cells in the cancer sample (e.g., tumor tissue sample) having an expression level of CLDN18.2 characterized by membrane staining intensity of at least 2+, and less than 70% of the cancer cells in the cancer sample (e.g., tumor tissue sample) having an expression level of CLDN18.2 characterized by membrane staining intensity of at least 2+ as measured using 14G11 on Leica Bond III LDT platform or an equivalent readout as measured on another test platform; and c) the low level of CLDN18.2 protein or CLDN18.2 expression corresponds to at least 1-10% (e.g., at least 1%, at least 5% or at least 10%) of the cancer cells in the cancer sample (e.g., tumor tissue sample) having an expression level of CLDN18.2 characterized by membrane staining intensity of at least 1+, and less than 40% of the cancer cells in the cancer sample (e.g., tumor tissue sample) having an expression level of CLDN18.2 characterized by membrane staining intensity of at least 2+ or 3+ as measured using 14G11 on Leica Bond III LDT platform, or an equivalent threshold as measured on another test platform or an equivalent threshold as measured with another anti-CLDN18.2 diagnostic antibody.

In another aspect, the present disclosure provides a method for treating a CLDN18.2-expressing cancer in a human subject in need thereof, comprising: administering to the subject a therapeutically effective amount of a CLDN18.2 antagonist in combination with chemotherapy and a PD-1/PD-L1 axis inhibitor, wherein the subject is determined to have high or low level of PD-L1 expression in a cancer sample (e.g., in a tumor tissue sample).

In another aspect, the present disclosure provides use of a CLDN18.2 antagonist in the manufacture of a medicament for treating a CLDN18.2-expressing cancer in a subject in need thereof, wherein the treatment comprises administering to the subject the medicament in combination with chemotherapy, wherein the subject is determined to have medium-to-high (e.g. medium or high), or low level of CLDN18.2 expression in a cancer sample (e.g., in a tumor tissue sample).

In another aspect, the present disclosure provides use of a CLDN18.2 antagonist in the manufacture of a medicament for treating a CLDN18.2-expressing cancer in a subject in need thereof, wherein the medicament further comprises chemotherapy, wherein the subject is determined to have medium-to-high (e.g. medium or high), or low level of CLDN18.2 expression in a cancer sample (e.g., in a tumor tissue sample).

In another aspect, the present disclosure provides a kit for use in identifying and treating a subject having a CLDN18.2-expressing cancer who may benefit from a treatment comprising a CLDN18.2 antagonist in combination with chemotherapy or with both chemotherapy and PD-1/PD-L1 axis inhibitor, comprising an anti-CLDN18.2 diagnostic reagent (e.g., anti-CLDN18.2 diagnostic antibody) and a package insert comprising instructions for using the treatment in a subject having medium-to-high (e.g. medium or high), or low level of the CLDN18.2 protein.

### BRIEF DESCRIPTION OF DRAWINGS

The drawings referenced herein form a part of the specification. Features shown in the drawing illustrate only some embodiments of the application, and not of all embodiments of the application, unless the detailed description explicitly indicates otherwise, and readers of the specification should not make implications to the contrary.

FIG. 1 shows study design of Cohort G from Transtar102 study (NCT04495296).

FIG. 2 shows Progression-Free Survival of Cohort G by CLDN18.2 level.

FIG. 3 shows efficacy percentage change in tumor size of Cohort C for patients in low expressors (≥10% tumor cell with staining 1+, and <40% with staining 2+ or 3+). n: efficacy population, Subjects with measurable lesions at baseline and at least one post-baseline tumor evaluation.

FIG. 4 shows efficacy percentage change in tumor size of Cohort G for patients in low expressors (≥10% tumor cell with staining 1+, and <40% with staining 2+ or 3+). n: efficacy population, Subjects with measurable lesions at baseline and at least one post-baseline tumor evaluation.

FIG. 5 shows efficacy percentage change in tumor size of Cohort G for patients in low expressors (o 10% tumor cell with staining 1+, and <40% with staining 2+ or 3+). n: efficacy population, Subjects with measurable lesions at baseline and at least one post-baseline tumor evaluation.

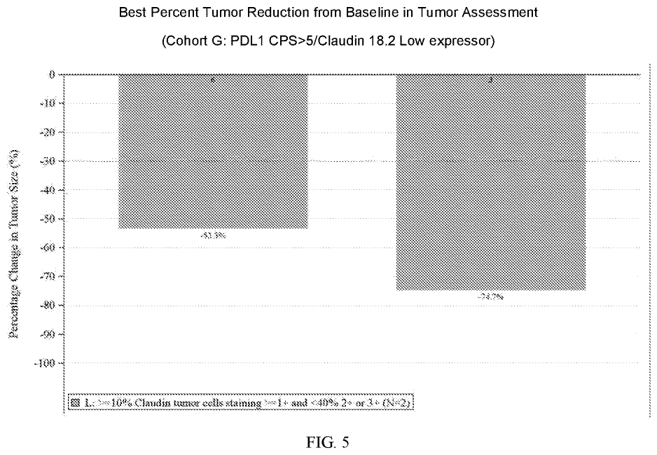

FIG. 6 shows summary of the CLDN18.2 and PD-L1 expression levels and overlapping across GC/GEJ cancer tissues.

FIG. 7 shows the IHC staining results of CLDN18.2 under light field by microscope.

FIG. 8 shows the IHC staining results of PD-L1 under light field by microscope.

FIG. 9A shows tumor growth curve of Hu18B10-HaLa in combination with Nivolumab and Oxaliplatin plus 5-FU inhibited GC PDX tumor growth (mean±S.E.M, n=8).

FIG. 9B shows distribution of tumor volumes on Day 22 of Hu18B10-HaLa in combination with Nivolumab and Oxaliplatin plus 5-FU inhibited GC PDX tumor growth (mean: S.E.M, n=8).

The same reference numbers will be used throughout the drawings to refer to the same or like parts.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### I. Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “CLDN18.2” refers to Claudin-18 splice variant 2 derived from mammals, such as primates (e.g. humans, monkeys) and rodents (e.g. mice). In certain embodiments, CLDN18.2 is human CLDN18.2. Exemplary sequence of human CLDN18.2 includes human CLDN18.2 protein (NCBI Ref Seq No. NP_001002026.1, or SEQ ID NO: 29). Exemplary sequence of CLDN18.2 includes Mus musculus (mouse) CLDN18.2 protein (NCBI Ref Seq No. NP_001181852.1), Macaca fascicularis (crab-eating macaque) CLDN18.2 protein (NCBI Ref Seq No. XP_015300615.1). CLDN18.2 is expressed in a cancer cell. In one embodiment said CLDN18.2 is expressed on the surface of a cancer cell.

As used herein, the term “antagonist” with respect to CLDN18.2 refers to any molecule that specifically binds to CLDN18.2 and/or partially or completely inhibits, blocks, or neutralizes a biological activity of CLDN18.2. Suitable CLDN18.2 antagonists may include, without limitation, antibodies, antisense oligonucleotides, peptides, and small organic molecules. In certain embodiments, the CLDN18.2 antagonist is an anti-CLDN18.2 antibody.

“Anti-CLDN18.2 antibody” as used herein refers to an antibody that is capable of specific binding to CLDN18.2 (e.g. human or non-human CLDN18.2) with a sufficient affinity, for example, to provide for diagnostic and/or therapeutic use.

The term “antibody” as used herein includes any immunoglobulin, monoclonal antibody, polyclonal antibody, multivalent antibody, bivalent antibody, monovalent antibody, multispecific antibody or bispecific antibody that binds to a specific antigen, or any polypeptides that mimics an antibody in terms of being capable of binding to a specific antigen. A native intact antibody comprises two heavy (H) chains and two light (L) chains. Mammalian heavy chains are classified as alpha, delta, epsilon, gamma, and mu, each heavy chain consists of a variable region (VH) and a first, second, and third constant region (CH1, CH2, CH3, respectively); mammalian light chains are classified as λ or κ, while each light chain consists of a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding domains disclosed herein may be defined or identified by the conventions of Kabat, IMGT, AbM, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); N. R. Whitelegg et al, Protein Engineering, v13(12), 819-824 (2000); Chothia, C. et al., Nature. December 21-28; 342(6252):877-83 (1989); Kabat E. A. et al., National Institutes of Health, Bethesda, Md. (1991); Marie-Paule Lefranc et al, Developmental and Comparative Immunology, 27: 55-77 (2003); Marie-Paule Lefranc et al, Immunome Research, 1(3), (2005); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant regions of the heavy and light chains are not involved in antigen-binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgAQ1 (alpha1 heavy chain), or IgA2 (alpha2 heavy chain). In certain embodiments, the antibody provided herein encompasses any antigen-binding fragments thereof.

As used herein, the term “antigen-binding fragment” or “antigen-binding portion” refers to a fragment (e.g., antibody fragment) formed from a fragment of an antibody comprising one or more CDRs, or any other portion (e.g., antibody portion) that binds to an antigen but does not comprise an intact native antibody structure. Examples of antigen-binding fragment/portion include, without limitation, a diabody, a Fab, a Fab′, a F(ab′)2, a Fd, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody. An antigen-binding fragment/portion is capable of binding to the same antigen to which the parent antibody binds. In certain embodiments, an antigen-binding fragment/portion may comprise one or more CDRs from a particular parent antibody.

“Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

“Fab′” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

“F(ab′)2” refers to a dimer of Fab′ that comprises two light chains and part of two heavy chains.

“Tv” with regard to an antibody refers to the smallest fragment of the antibody to bear the complete antigen binding site. A Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)). A “scFv dimer” refers to a single chain comprising two heavy chain variable regions and two light chain variable regions with a linker. In certain embodiments, an “scFv dimer” is a bivalent diabody or bivalent ScFv (BsFv) comprising VH-VL (linked by a peptide linker) dimerized with another VH-VL moiety such that VH's of one moiety coordinate with the VL's of the other moiety and form two binding sites which can target the same antigens (or eptipoes) or different antigens (or eptipoes). In other embodiments, a “scFv dimer” is a bispecific diabody comprising VH1-VL2 (linked by a peptide linker) associated with VL1-VH2 (also linked by a peptide linker) such that VH1 and VL1 coordinate and VH2 and VL2 coordinate and each coordinated pair has a different antigen specificity.

“Single-chain Fv-Fc antibody” or “scFv-Fe” refers to an engineered antibody consisting of a scFv connected to the Fc region of an antibody.

“Camelized single domain antibody,” “heavy chain antibody,” “nanobody” or “HCAb” refers to an antibody that contains two VH domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)). “Diabodies” include small antibody fragments with two antigen-binding sites, wherein the fragments comprise a VH domain connected to a VL domain in a single polypeptide chain (VH-VL or VL-VH) (see, e.g., Holliger P. et al., Proc Natl Acad Sci USA. July 15; 90(14):6444-8 (1993); EP404097; WO93/11161). The two domains on the same chain cannot be paired, because the linker is too short, thus, the domains are forced to pair with the complementary domains of another chain, thereby creating two antigen-binding sites. The antigen-binding sites may target the same of different antigens (or epitopes).

A “domain antibody” refers to an antibody fragment containing only the variable region of a heavy chain or the variable region of a light chain. In certain embodiments, two or more VH domains are covalently joined with a peptide linker to form a bivalent or multivalent domain antibody. The two VH domains of a bivalent domain antibody may target the same or different antigens.

In certain embodiments, a “(dsFv)2” comprises three peptide chains: two VH moieties linked by a peptide linker and bound by disulfide bridges to two VL moieties.

In certain embodiments, a “bispecific ds diabody” comprises VH1-VL2 (linked by a peptide linker) bound to VL1-VH2 (also linked by a peptide linker) via a disulfide bridge between VH1 and VL1.

In certain embodiments, a “bispecific dsFv” or “dsFv-dsFv’” comprises three peptide chains: a VH1-VH2 moiety wherein the heavy chains are bound by a peptide linker (e.g., a long flexible linker) and paired via disulfide bridges to VL1 and VL2 moieties, respectively. Each disulfide paired heavy and light chain has a different antigen specificity.

As used herein, a “bispecific” antibody refers to an artificial antibody which has fragments derived from two different monoclonal antibodies and is capable of binding to two different epitopes. The two epitopes may present on the same antigen, or they may present on two different antigens.

The term “humanized” as used herein means that the antibody or antigen-binding fragment comprises CDRs derived from non-human animals, FR regions derived from human, and when applicable, constant regions derived from human. In certain embodiments, the amino acid residues of the variable region framework of the humanized CLDN18.2 antibody are substituted for sequence optimization. In certain embodiments, the variable region framework sequences of the humanized CLDN18.2 antibody chain are at least 65%, 70%, 75%, 80%, 85%, 90%, 95% or 100% identical to the corresponding human variable region framework sequences.

The term “chimeric” as used herein refers to an antibody or antigen-binding fragment that has a portion of heavy and/or light chain derived from one species, and the rest of the heavy and/or light chain derived from a different species. In an illustrative example, a chimeric antibody may comprise a constant region derived from human and a variable region derived from a non-human species, such as from mouse.

The term “affinity” as used herein refers to the strength of non-covalent interaction between an immunoglobulin molecule (i.e., antibody) or fragment thereof and an antigen.

The term “specific binding” or “specifically binds” as used herein refers to a non-random binding reaction between two molecules, such as for example between an antibody and an antigen. In certain embodiments, the antibodies or antigen-binding fragments provided herein specifically bind to human and/or non-human CLDN18.2 with a binding affinity (KD) of ≤10⁻⁶ M (e.g., <5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤4×10⁻⁹ M, 3×10⁻⁹M, ≤2×10⁻⁹ M, or ≤10⁻⁹ M. KD used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the KD value can be appropriately determined by using flow cytometry method. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least 10 to 100 times over the background.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum correspondence. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm. In certain embodiments, the non-identical residue positions may differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference.

As used herein, a “homologue sequence” refers to a polynucleotide sequence (or its complementary strand) or an amino acid sequence that has sequence identity of at least 80% (e.g. at least 85%, 88%, 90%, 91%, 92%, 93%, 94%, 95%, 96%, 97%, 98%, 99%) to another sequence when optionally aligned.

The term “subject” includes human and non-human animals. Non-human animals include all vertebrates, e.g., mammals and non-mammals, such as non-human primates, mouse, rat, cat, rabbit, sheep, dog, cow, chickens, amphibians, and reptiles. Except when noted, the terms “patient” or “subject” are used herein interchangeably.

“Effector functions” or “antibody effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and antibody dependent cell mediated phagocytosis (ADCP), where nonspecific cytotoxic cells that express FcγRs recognize bound antibody on a target cell and subsequently cause phagocytosis of the target cell. Effector functions include both those that operate after the binding of an antigen and those that operate independent of antigen binding.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

“Cancer” as used herein refers to any medical condition characterized by malignant cell growth or neoplasm, abnormal proliferation, infiltration, or metastasis, and includes both solid tumors and non-solid cancers (e.g., hematologic malignancies) such as leukemia. As used herein “solid tumor” refers to a solid mass of neoplastic and/or malignant cells.

The term “therapeutically effective amount” or “effective amount” means the amount of a pharmaceutical agent that produces some desired local or systemic therapeutic effect at a reasonable benefit/risk ratio applicable to any treatment alone or together with further doses. In the case of the treatment of a particular disease, the desired local or systemic therapeutic effect preferably relates to inhibition of the course of the disease. This comprises slowing down the progress of the disease and, in particular, interrupting or reversing the progress of the disease.

When administered for preventing a disease, the amount is sufficient to avoid or delay onset of the disease. A therapeutically effective amount or an effective amount need not be curative or prevent a disease or condition from ever occurring. An effective amount of the pharmaceutical agent described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the pharmaceutical agent described herein may depend on various of such parameters. In the case that a reaction in a patient is insufficient with an initial dose, higher doses (or effectively higher doses achieved by a different, more localized route of administration) may be used. In certain embodiments, a therapeutically effective amount of a pharmaceutical agent will depend on its therapeutic index, solubility, and the like.

The term “survival”, as used herein, refers to the subject remaining alive, which can be indicated by progression free survival (PFS) and/or overall survival (OS). Survival can be estimated by the Kaplan-Meier method. Differences in survival are computed using the stratified log-rank test.

As used herein, the term “staining” refers to binding a target (e.g., an antigen) in a biological sample (e.g., cancer sample, such as tumor tissue sample) with a target-specific binding agent (e.g., a diagnostic antibody or a nucleic acid) and then detecting the presence of the target-specific binding agent on the cells of the biological sample using a detectable label or chromogen. The detectable label can be directly conjugated to the target-specific binding agent (e.g., a primary antibody) or may be conjugated to a secondary reagent that binds specifically to an unlabeled target-specific reagent (e.g., a secondary antibody). In some cases, the target-specific reagent is itself detectable, and thus no additional attached label is needed.

As used herein, the term “target-specific binding agent” means any agent that specifically binds to an analyte of interest, e.g., a target of interest that is present in a tissue section (e.g., tumor tissue section), such as a polypeptide or polynucleotide. Exemplary target-specific binding agents include antibodies, receptors, and ligands, or target-binding fragments thereof, polynucleotide probes, and such.

As used herein, the term “chromogen” refers to a substance that can be converted into a colored compound under specific conditions, for example, when acted upon by an enzyme or under specific chemical/reaction conditions.

As used herein, the terms “primary antibody” and “secondary antibody” refer to different antibodies, where a primary antibody is a polyclonal or monoclonal antibody from one species (rabbit, mouse, goat, donkey, etc.) that specifically recognizes an antigen (e.g., a biomarker, such as CLDN18.2) in a biological sample (e.g., a cancer sample, such as a tumor tissue sample) under study, and a secondary antibody is an antibody (usually polyclonal) from a different species that specifically recognizes the primary antibody, for example, in its Fc region.

It is noted that in this disclosure, terms such as “comprises”, “comprised”, “comprising”, “contains”, “containing” and the like are intended to be inclusive or open-ended, and do not exclude additional, un-recited elements or method steps.

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

### II. Combination Therapy for CLDN18.2-Expressing Cancers

The present disclosure provides a method for treating a CLDN18.2-expressing cancer in a human subject in need thereof. In certain embodiments, the subject is determined to have medium-to-high (e.g. medium, or high), or low level of CLDN18.2 expression in a cancer sample (e.g., in a tumor tissue sample). In certain embodiments, the method comprises administering to the subject a therapeutically effective amount of a CLDN18.2 antagonist in combination with chemotherapy. In certain embodiments, the method further comprises administering an immunotherapy.

In another aspect, the present disclosure provides use of a CLDN18.2 antagonist in the manufacture of a medicament for treating a CLDN18.2-expressing cancer in a subject in need thereof. In certain embodiments, the subject is determined to have medium-to-high (e.g. medium, or high), or low level of CLDN18.2 expression in a cancer sample (e.g., in a tumor tissue sample). In certain embodiments, the treatment comprises administering to the subject the medicament in combination with chemotherapy. In certain embodiments, the treatment further comprises administering an immunotherapy. In certain embodiments, the medicament further comprises chemotherapy and/or immunotherapy.

In another aspect, the present disclosure also provides a method of identifying and treating a subject having a CLDN18.2-expressing cancer who may benefit from a treatment comprising a CLDN18.2 antagonist in combination with chemotherapy. In certain embodiments, the treatment further comprises immunotherapy. In certain embodiments, the method comprises determining the level of CLDN18.2 protein in a cancer sample (e.g., in a tumor tissue sample). In certain embodiments, the method further comprises selecting a subject having medium-to-high (e.g. medium, or high), or low level of the CLDN18.2 protein for the treatment described above.

a. Subject with CLDN18.2 Expression

In certain embodiments, the subject is determined to have medium-to-high (e.g. medium, or high) level of CLDN18.2 expression in a cancer sample. In certain embodiments, the subject is determined to have low level of CLDN18.2 expression in a cancer sample. In certain embodiments, the subject is human.

As used herein, the term “low level of CLDN18.2 expression”, used interchangeably with the term “low level of the CLDN18.2 protein” is intended to mean a detectable level of CLDN18.2 expression on the surface of the cancer cells of the subject, which level is relatively lower than a predetermined threshold.

The predetermined threshold can be determined by a skilled person in the art, for example, as a threshold for moderate or medium expression level of CLDN18.2 protein for a specific type of cancer cell. Therefore, a level lower than the predetermined threshold for moderate or medium expression level would mean low level of the CLDN18.2 protein.

The threshold for moderate or medium expression level the CLDN18.2 protein can be determined in a population of samples, cells, tumors, or cancers of a certain cancer type, by determining actual expression levels of CLDN18.2 protein in the population, and ranking the population by the actual expression levels. For example, in a population having cancer (such as gastric and gastro-oesophageal adenocarcinoma), a moderate or medium expression level of the CLDN18.2 protein in that cancer type may be the 70^{th} percentile, 65^{th} percentile, 60^{th} percentile, the 55^{th} percentile, the 50^{th} percentile, the 45^{th} percentile, the 40^{th}, the 35^{th}, the 30^{th}, the 25^{th} or the 20^{th} percentile of the expression level of CLDN18.2 protein in such population. Such actual expression levels can be quantified in an assay under specified assay conditions, such as an immuno-histochemistry (IHC) assay, or a hybridization assay, or an amplification assay such as quantitative reverse transcription polymerase chain reaction (RT-PCR) assay.

In certain embodiments, a low level of the CLDN18.2 protein in a specific cancer can be lower than the 70^{th} percentile, the 65^{th} percentile, lower than the 60^{th} percentile, lower than the ₅₅^{th} percentile, lower than the 50^{th} percentile, lower than the 45^{th} percentile, lower than the 40th percentile, lower than the 35^{th} percentile, lower than the 30^{th} percentile, lower than the 25th percentile, or lower than the 20^{th} percentile in a given population of the specific cancer.

As used herein, the term “medium-to-high level of CLDN18.2 expression”, used interchangeably with the term “medium-to-high level of the CLDN18.2 protein” is intended to mean a level of CLDN18.2 expression on the surface of the cancer cells of the subject, which level is relatively higher than or comparable to a predetermined threshold for moderate or medium expression level.

The threshold for moderate or medium expression level of CLDN18.2 protein can also be determined by the expression level as measured by a suitable assay in the cancer sample (e.g., tumor tissue sample). Various suitable methods are known in the art, including, without limitation, protein-based assays such as quantitative fluorescence cytometry, immunohistochemistry (IHC) methods and enzyme-linked immunosorbent assay (ELISA), or nucleic acid-based assays such as amplification assays, hybridization assays or sequencing assays.

A skilled person in the art would understand that, depending on the type of assay and the specific conditions of the assay, the absolute value representing the expression level of CLDN18.2 in a specific sample can vary. Accordingly, the predetermined threshold for moderate or medium expression level of CLDN18.2 protein for a specific cancer may also vary depending on type of assay and the specific conditions of the assay, and can be determined by a skilled person in the art, taking consideration of a variety of factors, including for example, the type of sample, the type of assay and assay conditions, the specific type of cancer or tumor, the CLDN18.2-diagnostic antibody to be used, and/or the type of the treatment, to name a few.

In certain embodiments, the expression level of CLDN18.2 can be measured by IHC assay using a CLDN18.2 diagnostic antibody and can be represented by membrane staining intensity. In other words, the level of CLDN18.2 protein or the level of CLDN18.2 expression can be quantified by membrane staining intensity measured by IHC using an anti-CLDN18.2 diagnostic reagent (e.g., anti-CLDN18.2 diagnostic antibody, e.g., 14G11 as described below). Membrane staining intensity can range from, for example, 0 (no staining), 1+ (weak staining), 2+ (distinct staining), 3+ (strong staining) and 4+ (extremely strong/saturated signal).

In certain embodiments, the CLDN18.2 expression level can be quantified based on the percentage of cells (e.g. cancer cells) showing certain level of membrane staining intensity in the sample. In certain embodiments, the CLDN18.2 expression level can be quantified by percentage of cancer cells having a predetermined threshold of the membrane staining intensity measured by IHC using an anti-CLDN18.2 diagnostic reagent (e.g., anti-CLDN18.2 diagnostic antibody, e.g., 14G11 as described below). For example, to assess CLDN18.2 expression in a sample, an observer can examine the number of cells stained positive for CLDN18.2 in one or more selected field(s) under a microscope, and calculate or estimate the percentage of cells (e.g. cancer cells) that shows certain level of membrane staining intensity for CLDN18.2. In certain embodiments, viable cells are stained and analyzed for expression level of claudin 18.2.

In certain embodiments, the CLDN18.2 expression level can also be quantified based on intensity scores calculated based on the membrane staining intensity and the percentage of cells having certain level of membrane staining intensity. For example, an intensity score such as 4-point HSCORE can be calculated based on intensity of staining ranging from 0 (no staining), 1+ (weak staining), 2+ (distinct staining), 3+ (strong staining) and 4+ (extremely strong/saturated signal) and multiplying by the percent of cells staining at each intensity (0 to 100%) (see details in, McCarty, K. S. Jr, et al, Cancer Res. 46(suppl 8):4244s-4248s (1986)). For another example, Alfred score can be calculated based on a Total Score (TS, range 0 to 8) by adding together a proportion score (PS) and an intensity score (IS). PS is the proportion of positive tumor cells ranging from 0 to 5 (0=no positive cells, 1=1/100 cells are positive, 2=1/10 cells are positive, 3=1/3 cells are positive, 4=2/3 of cells are positive, 5=all tumor cells are positive). IS means the average staining intensity of positive tumor cells ranging from 0 to 3 (0=negative, 1=weak, 2=intermediate staining, 3=strong staining) (see, details in, Alfred D C et al. Mod Pathol. 11: 155-168 (1998)).

In certain embodiments, the expression level of CLDN18.2 is measured by IHC assay.

Briefly, the cancer sample (e.g., tumor tissue sample, optionally a formalin-fixed paraffin-embedded (FFPE) tissue sample) having a certain section thickness (e.g., 1-5 um (e.g., 2-5 um, 3-5 um or 4-5 um) is subjected to antigen retrieval in a certain retrieval solution (e.g., BOND Epitope Retrieval Solution 2 (Leica, AR9640), BOND Epitope Retrieval Solution 2 (Leica, AR9640) or CC1 Solution (Ventana, 760-700)) that is compatible with the staining system (e.g., Leica Bond III LDT platform, Dako Autostainer Link 48 platform or VENTANA BenchMark XT/ULTRA platform) for a certain period of time (e.g., 15-120 min, 25-100 min, 35-90 min, 45-80 min, 55-90 min, 65-80 min, 70 min, 15-30 min, 20-30 min, or 64-104 min) at a certain temperature (e.g., 50° C.-100° C., 60° C.-100° C., 70° C.-100° C., 80° C.-100° C., 90° C.-100° C., 92° C., 94° C., 96° C., 97° C., 98° C. or 100° C.). After antigen retrieval, the sample is contacted (e.g., incubated) with an anti-CLDN18.2 diagnostic antibody (e.g., 14G11 as described below) at a certain concentration (e.g., 0.05-20 ug/ml, 0.1-15 ug/ml, 0.15-10 ug/ml, 0.15-5 ug/ml, 0.2-10 ug/ml, 0.3-10 ug/ml, 0.4-10 ug/ml, 0.5-10 ug/ml, 0.6-10 ug/ml, 0.7-10 ug/ml, 0.8-10 ug/ml, 0.9-10 ug/ml, 1-10 ug/ml or 0.15-5 ug/ml) for a certain period of time (e.g., 10-60 min, 15-50 min, 15-40 min, 15-35 min, 15-30 min, 16-32 min or 20-30 min). This can be followed by contacting (e.g., incubating) the sample with a secondary antibody (e.g., BOND Polymer Detection (Leica, DS9800), FLEX/HRP reagent (Dako, K8002), or OptiView HRP Multimer reagent (Ventana, 760-700)) and visualizing in a staining system (e.g., Leica Bond III LDT platform, Dako Autostainer Link 48 platform or VENTANA BenchMark XT/ULTRA platform). The skilled person would understand that the section thickness, the certain period of time, the certain temperature and the certain concentration of anti-CLDN18.2 diagnostic antibody may vary depending on the imaging system and diagnostic antibody used.

Different assay format can be used, including for example, IHC assay by Leica autostainer (e.g. Leica Bond III LDT platform), IHC assay by Dako autostainer (e.g. Dako Autostainer Link 48 platform), or IHC assay by VENTANA autostainer (e.g. VENTANA BenchMark XT/ULTRA platform).

For Leica autostainer (e.g. Leica Bond III LDT platform), the cancer sample (e.g., tumor tissue sample, or formalin-fixed paraffin-embedded (FFPE) tumor tissue sample) is sectioned to have a thickness of 4-5 um, and subject to antigen retrieval for 15-30 minutes at 100° C. Antigen retrieval is performed using BOND Epitope Retrieval Solution 2 (Leica, AR9640), and detected by the CLDN18.2 diagnostic reagent (e.g., 14G11 as described below) at a concentration range of 0.15-5 pg/mL which is allowed to contact with the cancer sample (e.g., tumor tissue sample) for 15-30 minutes. More specific description for Leica Bond III LDT platform is provided in Example 1.

In certain embodiments, when the Leica autostainer (e.g. Leica Bond III LDT platform) is used for the IHC assay, the predetermined threshold for moderate or medium expression level expression level of CLDN18.2 protein is 30%, 31%, 32%, 33%, 34%, 35%, 36%, 37%, 38%, 39%, 40%, 41%, 42%, 43%, 44%, 45%, 46%, 47%, 48%, 49% or 50% of the cancer cells in the cancer sample (e.g., tumor tissue sample) having membrane staining intensity of at least 2+ or at least 3+. In such embodiments, the low level of the CLDN18.2 protein is lower than 30%, or lower than 31%, lower than 32%, lower than 33%, lower than 34%, lower than 35%, lower than 36%, lower than 37%, lower than 38%, lower than 39%, lower than 40%, or lower than 41%, lower than 42%, lower than 43%, lower than 44%, lower than 45%, lower than 46%, lower than 47%, lower than 48%, lower than 49%, lower than 50% of the cancer cells in the cancer sample (e.g., tumor tissue sample) having membrane staining intensity of at least 2+ or at least 3+. In certain embodiments, the low level of the CLDN18.2 protein corresponds to at least 1%, at least 2%, at least 3%, at least 4%, at least 5%, at least 6%, at least 7%, at least 8%, at least 9%, or at least 10% of the cancer cells in the cancer sample (e.g., tumor tissue sample) having membrane staining intensity of at least 1+.

In certain embodiments, the low level of CLDN18.2 protein or CLDN18.2 expression corresponds to at least 1% (e.g., at least 2%, at least 5% or at least 10%) of the cancer cells in the cancer sample (e.g., tumor tissue sample) having an expression level of CLDN18.2 characterized by membrane staining intensity of at least 1+, and less than 40% of the cancer cells in the cancer sample (e.g., tumor tissue sample) having an expression level of CLDN18.2 characterized by membrane staining intensity of at least 2+ (e.g. 2+ or 3+) as measured using an anti-CLDN18.2 diagnostic antibody (e.g. antibody 14G11), optionally on Leica autostainer (e.g. Leica Bond III LDT platform). The low level of CLDN18.2 protein or CLDN18.2 expression can also be characterized with an equivalent readout using the same anti-CLDN18.2 diagnostic antibody (e.g. antibody 14G11) or a different anti-CLDN18.2 diagnostic antibody, as measured on another test platform (for example, on Dako Autostainer Link 48 platform or on VENTANA BenchMark XT/ULTRA platform).

In certain embodiments, the medium-to-high level of CLDN18.2 protein or CLDN18.2 expression corresponds to at least 40% of the cancer cells in the cancer sample (e.g., tumor tissue sample) having an expression level of CLDN18.2 characterized by membrane staining intensity of at least 2+ (e.g. 2+ or 3+), as measured using an anti-CLDN18.2 diagnostic antibody (e.g. antibody 14G11), optionally on Leica autostainer (e.g. Leica Bond III LDT platform). The medium-to-high level of CLDN18.2 protein or CLDN18.2 expression can also be characterized with an equivalent readout using the same anti-CLDN18.2 diagnostic antibody (e.g. antibody 14G11) or a different anti-CLDN18.2 diagnostic antibody, as measured on another test platform.

In certain embodiments, the group of subjects having medium-to-high level of CLDN18.2 protein or CLDN18.2 expression can be further divided into two subgroups, namely, having a medium level of CLDN18.2 protein or CLDN18.2 expression, or a high level of CLDN18.2 protein or CLDN18.2 expression.

In certain embodiments, the high level of CLDN18.2 protein or CLDN18.2 expression corresponds to at least 70% of the cancer cells in the cancer sample (e.g., tumor tissue sample) having an expression level of CLDN18.2 characterized by membrane staining intensity of at least 2+ (e.g., 2+ or 3+) as measured using an anti-CLDN18.2 diagnostic antibody (e.g. antibody 14G11), optionally on Leica autostainer (e.g. Leica Bond III LDT platform). The high level of CLDN18.2 protein or CLDN18.2 expression can also be characterized with an equivalent readout using the same anti-CLDN18.2 diagnostic antibody (e.g. antibody 14G11) or a different anti-CLDN18.2 diagnostic antibody, as measured on another test platform.

In certain embodiments, the medium level of CLDN18.2 protein or CLDN18.2 expression corresponds to at least 40% of the cancer cells in the cancer sample (e.g., tumor tissue sample) having an expression level of CLDN18.2 characterized by membrane staining intensity of at least 2+ (e.g., 2+ or 3+), and less than 70% of the cancer cells in the cancer sample (e.g., tumor tissue sample) having an expression level of CLDN18.2 characterized by membrane staining intensity of at least 2+ (e.g., 2+ or 3+) as measured using an anti-CLDN18.2 diagnostic antibody (e.g. antibody 14G11), optionally on Leica autostainer (e.g. Leica Bond III LDT platform). The medium level of CLDN18.2 protein or CLDN18.2 expression can also be characterized with an equivalent readout using the same anti-CLDN18.2 diagnostic antibody (e.g. antibody 14G11) or a different anti-CLDN18.2 diagnostic antibody, as measured on another test platform.

In addition to Leica autostainer (e.g. Leica Bond III LDT platform), other suitable platforms can also be used to measure the expression level of the CLDN18.2 protein. A reference standard or calibration method may be employed to establish equivalency between the readouts obtained from different platforms. For instance, while the Leica Bond III LDT platform may classify a sample as having membrane staining intensity of 1+, another assay conducted on a different platform may yield another measurement of an expression level. Through appropriate calibration and comparison studies, it can be demonstrated that these differing readouts correspond to equivalent thresholds for the expression level of CLDN18.2 protein. This ensures that despite variations in the readout values, different assays provide consistent and comparable measures for the same sample.

For Dako Autostainer Link 48 platform, the cancer sample (e.g., tumor tissue sample) is sectioned to have a thickness of 4 um, and subject to antigen retrieval for 20-30 minutes at 97-100° C. Antigen retrieval is performed using Target Retrieval Solution, High pH (Dako, K8002), and detected by the CLDN18.2 diagnostic reagent at a concentration range of 0.5-10 pg/mL which is allowed to contact with the cancer sample (e.g., tumor tissue sample) for 20-30 minutes. In an illustrative example, IHC can be performed on slides of 4% neutral buffered formalin fixed paraffin embedded samples. Sections can be cut at a thickness of 4 μm and mounted onto positive charged slides. In pre-set PT Link pre-treatment module (Dako, PT100/PT101/PT200), all slides can be proceeded to deparaffinization, rehydration and antigen retrieval following 3-in-1 procedure by heating in Target Retrieval Solution, High pH (Dako, K8002) for 20-30 minutes at 97-100° C. After washing in PT Link Rinse Station (Dako, PT109), the slides are placed on the Dako Autostainer Link 48 platform (Dako, AS480) with preprogrammed staining protocol: quench, block with Peroxidase Block Reagent for 5 minutes and incubated with the diagnostic antibody at dynamic range of concentration (0.5-10 pg/mL) diluted in Antibody Diluent (Dako, S0809) for 20-30 minutes. Antibody binding was amplified by FLEX+ Mouse (LINKER) and visualized with FLEX/HRP (Dako, K8002) to formation of a brown precipitates by reaction with DAB substrate. Sections were finally counterstained with Hematoxylin and mounted with permanent mounting medium. IHC result can be scored by board-certified pathologists under light field by microscope based on membrane staining intensity (neg (0), weak (1+), moderate (2+), strong (3+)) as well as Tumor Proportion Score (TPS) defined as the percentage of viable invasive tumor cells showing ≥1+ target staining intensity among all viable invasive tumor cells. Normal and/or adjacent uninvolved tissues are not included in the scoring assessment.

In certain embodiments, cancer cells that express a low level of the CLDN18.2 protein are not sufficient to respond significantly to a single-agent treatment of an anti-claudin 18.2 antibody, for example, the single-agent treatment of anti-claudin 18.2 antibody is Zolbetuximab. In certain embodiments, cancer cells that express a low level of the CLDN18.2 protein are not sufficient to respond significantly to a combination therapy involving an anti-claudin 18.2 antibody, for example, the anti-claudin 18.2 antibody is Zolbetuximab. The combination therapy can comprise chemotherapy and/or immunotherapy.

a-i). Anti-CLDN18.2 Diagnostic Reagent

In some embodiments, the anti-CLDN18.2 diagnostic reagent is a nucleic acid or nucleic acid binding agent, for example, as those employed in in situ hybridization (ISH, e.g., fluorescence-in-situ-hybridization (FISH)) reactions. For example, the anti-CLDN18.2 diagnostic reagent can be a RNA, DNA, DNA/RNA hybrid molecule, and/or peptide nucleic acid (PNA).

In certain embodiments, the anti-CLDN18.2 diagnostic reagent comprises an anti-CLDN18.2 diagnostic antibody. The anti-CLDN18.2 diagnostic antibody can be any anti-CLDN18.2 antibodies that can sensitively detect the expression of CLDN18.2 on a cancer sample (e.g., tumor tissue sample). The anti-CLDN18.2 diagnostic antibody can be a monoclonal antibody, polyclonal antibody or fragment of thereof. For example, the anti-CLDN18.2 diagnostic antibody can be those described in PCT/CN2021/095411. In certain embodiments, the anti-CLDN18.2 diagnostic antibody comprises HCDR1, HCDR2 and HCDR3 and/or LCDR1, LCDR2 and LCDR3 sequences of 14G11. As used herein, the term “14G11” refers to an anti-CLDN18.2 antibody comprising a heavy chain variable region comprising an amino acid sequence of SEQ ID NO: 21, and a light chain variable region comprising an amino acid sequence of SEQ ID NO: 22. In certain embodiments, the anti-CLDN18.2 diagnostic antibody is 14G11, which comprises a heavy chain variable region comprising an amino acid sequence of SEQ ID NO: 21, and a light chain variable region comprising an amino acid sequence of SEQ ID NO: 22. In certain embodiments, the anti-CLDN18.2 diagnostic antibody is 14G11, which comprises a heavy chain comprising an amino acid sequence of SEQ ID NO: 19, and a light chain comprising an amino acid sequence of SEQ ID NO: 20. The anti-CLDN18.2 diagnostic antibody can also be other commercially available anti-CLDN18.2 antibodies, such as [EPR19202] (ab222512), 43-14A, SDT-102-24, MIL 93 or 3B10.

The anti-CLDN18.2 diagnostic reagent described herein may be attached to any suitable detectable label or enzyme capable of producing a detectable label. Any convenient enzymatic label/chromogen deposition system can be employed (e.g., as used in standard IHC methods). In some embodiments, the detectable label is a fluorescent tag.

In certain embodiments, the label is indirectly conjugated with the anti-CLDN18.2 diagnostic antibody. The skilled person will be aware of various techniques for achieving this. For example, the anti-CLDN18.2 diagnostic antibody can be conjugated with biotin and the label can be conjugated with avidin, or vice versa. Biotin binds selectively to avidin and thus, the label can be conjugated with the antibody in such indirect manner. Alternatively, to achieve indirect conjugation of the label with the antibody, the antibody can be conjugated with a small hapten and the label can be conjugated with an anti-hapten antibody. As such, indirect conjugation of the label with the antibody can be achieved. In certain embodiments, one or more secondary antibody molecules may be conjugated with a label-conjugated polymer backbone, such that amplification of the signal may be achieved.

Indirectly and directly labeled secondary antibodies are also commercially available, such as BOND Polymer Detection (Leica, DS9800), FLEX/HRP reagent (Dako, K8002), and OptiView HRP Multimer reagent (Ventana, 760-700).

a-ii) Sample

In certain embodiments, the subject is or has been determined to have medium-to-high, or low level of CLDN18.2 expression or low level of the CLDN18.2 protein in the cancer sample (e.g., tumor tissue sample), derived from the subject. In certain embodiments, the method provided herein further comprises providing a biological sample from the subject. Any biological sample suitable for conducting the methods provided herein can be obtained from the subject. As used herein, “biological sample” refers to a biological specimen taken by sampling from a subject, optionally with additional processing. The collection of a sample from a subject is performed in accordance with the standard protocol generally followed by hospital or clinics, such as during a biopsy.

In certain embodiments, the biological sample comprises a cancer sample (e.g., tumor tissue sample). In some embodiments, the biological sample is a fresh or archived sample obtained from a tumor tissue, e.g., by a tumor biopsy or fine needle aspirate. In some embodiments, the sample can be any biological fluid containing cancer cells or non-cancer cells (e.g. peripheral blood mononuclear cells (PBMC)).

Examples of a biological sample include without limitation, bodily fluid, such as blood, plasma, serum, urine, vaginal fluid, uterine or vaginal flushing fluids, pleural fluid, ascetic fluid, cerebrospinal fluid, saliva, sweat, tears, sputum, bronchioalveolar lavage fluid, etc., and tissues, such as biopsy tissue (e.g. biopsied bone tissue, bone marrow, breast tissue, gastrointestinal tract tissue, lung tissue, colon tissue, liver tissue, prostate tissue, brain tissue, nerve tissue, meningeal tissue, colon tissue, renal tissue, endometrial tissue, cervical tissue, lymph node tissue, muscle tissue, or skin tissue), a paraffin embedded tissue. In a further embodiment, a biological sample comprises cells, tissue, blood, plasma, serum, urine, mouthwash, stool, saliva, and any combination thereof.

In certain embodiments, the biological sample (e.g., cancer or tumor tissue sample) may be further processed to, for example, isolate the analyte such as the nucleic acids or proteins. For example, the biological sample (e.g., cancer or tumor tissue sample) from the subject can be exposed to anti-CLDN18.2 diagnostic reagent, which binds to and detects the translated CLDN18.2 mRNA or the expressed CLDN18.2 protein.

In certain embodiments, the sample is a fixed tissue sample, optionally a formalin-fixed paraffin-embedded (FFPE) tissue sample.

FFPE tissue sections can be of about 3-4 millimeters, and preferably 4-40 micrometers, which are mounted and dried on a microscope slide. Examples of paraffin include, but are not limited to, Paraplast, Broloid and Tissue.g emay. For a fixed tissue sample such as an FFPE tissue sample, the sample may be deparaffinized before contacting with the anti-CLDN18.2 antibody or antigen-binding fragment thereof provided herein.

In some embodiments, the deparaffinized sample may be further treated to allow antigen retrieval. Antigen retrieval refers to any technique in which the masking of an epitope is reversed and epitope-antibody binding is restored. The need for antigen retrieval depends on multiple variables, including but not limited to, the target antigen, the antibody used, the type of tissue, and the method and duration of fixation. Techniques of antigen retrieval generally include protease-induced epitope retrieval (PIER, by using enzymes such as Proteinase K, Trypsin, and/or Pepsin) and heat-induced epitope retrieval (HIER, by using microwave ovens, pressure cookers, vegetable steamers, autoclaves, or water baths).

In certain embodiments, the sample can be further processed by a desirable method for determining expression level of the at least one biomarker, such as CLDN18.2.

In certain embodiments, the level of CLDN18.2 expression or the CLDN18.2 protein is determined from the cancer sample (e.g., tumor tissue sample).

b. The CLDN18.2 Antagonist

The CLDN18.2 antagonist used in the methods provided herein can comprise an anti-CLDN18.2 antibody or antigen-binding fragment thereof. The anti-CLDN18.2 antibody can be a monoclonal antibody, polyclonal antibody, humanized antibody, chimeric antibody, recombinant antibody, bispecific antibody, labeled antibody, bivalent antibody, or anti-idiotypic antibody.

In certain embodiments, the anti-CLDN18.2 antibody comprises heavy chain HCDR1, HCDR2 and HCDR3 and/or light chain LCDR1, LCDR2 and LCDR3 sequences, wherein:
the HCDR1 sequence comprises GYNMN (SEQ ID NO: 1), or a homologue sequence of at least 80% sequence identity thereof;
the HCDR2 sequence comprises NIDPYYGGTSYNQKFKG (SEQ ID NO: 2), or a homologue sequence of at least 80% sequence identity thereof;
the HCDR3 sequence comprises MYHGNAFDY (SEQ ID NO: 3), or a homologue sequence of at least 80% sequence identity thereof;
the LCDR1 sequence comprises KSSQSLLNSGNLKNYLT (SEQ ID NO: 4) or a homologue sequence of at least 80% sequence identity thereof;
the LCDR2 sequence comprises WASTRKS (SEQ ID NO: 5) or a homologue sequence of at least 80% sequence identity thereof;
the LCDR3 sequence comprises QNDYSYPLT (SEQ ID NO: 6) or a homologue sequence of at least 80% sequence identity thereof.

CDRs are known to be responsible for antigen binding, however, it has been found that not all of the 6 CDRs are necessarily indispensable or unchangeable. In other words, it is possible to replace or change or modify 1, 2, or 3 CDRs in the anti-CLDN18.2 antibody, yet substantially retain the specific binding affinity to CLDN18.2.

In certain embodiments, the anti-CLDN18.2 antibody comprises a heavy chain CDR3 sequence of MYHGNAFDY (SEQ ID NO: 3). Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In some embodiments, the anti-CLDN18.2 antibody comprises all or a portion of the heavy chain variable domain and/or all or a portion of the light chain variable domain. In one embodiment, the anti-CLDN18.2 antibody is a single domain antibody which consists of all or a portion of the heavy chain variable domain provided herein. More information of such a single domain antibody is available in the art (see, e.g., U.S. Pat. No. 6,248,516).

In certain embodiments, the anti-CLDN18.2 antibody comprises a heavy chain variable region and a light chain variable region, wherein the heavy chain variable region comprises an amino acid sequence of SEQ ID NO: 7, and the light chain variable region comprises an amino acid sequence of SEQ ID NO: 8.

In certain embodiments, the anti-CLDN18.2 antibody further comprises an immunoglobulin constant region, optionally a constant region of human Ig, or optionally a constant region of human IgG. In certain embodiments, the anti-CLDN18.2 antibody further comprises a constant region of human IgG1, IgG2, IgG3, or IgG4.

In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises CK or Ck.

In certain embodiments, the anti-CLDN18.2 antibodies and antigen-binding fragments thereof provided herein comprises a constant region of IgG1 isotype. In certain embodiments, the constant region of human IgG1 comprises SEQ ID NO: 9, or a homologous sequence having at least 80% (e.g. at least 85%, 90%, 95%, 96%, 97%, 98%, or 99%) sequence identity thereof.

Constant region of IgG1 isotype can induce effector functions such as ADCC or CDC. Effector functions of the anti-CLDN18.2 antibody can lead to cytotoxicity to cells expressing CLDN18.2. Effector functions can be evaluated using various assays such as Fc receptor binding assay, C1q binding assay, and cell lysis assay, and any of the assays described above for determining ADCC or CDC.

In certain embodiments, the constant region comprises one or more amino acid residue substitutions or modifications conferring increased CDC or ADCC relative to wild-type constant region.

As used herein, “ADCC” or “antibody-dependent cell-mediated cytotoxicity” refers to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. Lysis of the target cell is extracellular, requires direct cell-to-cell contact, and does not involve complement. ADCC can be viewed as a mechanism to directly induce a variable degree of immediate tumor destruction that leads to antigen presentation and the induction of tumor-directed T-cell responses. In vivo induction of ADCC is believed to lead to tumor-directed T-cell responses and host-derived antibody responses.

In certain embodiments, the constant region comprises one or more amino acid residue substitutions relative to SEQ ID NO: 9, selected from the group consisting of: L235V, F243L, R292P, Y300L, P396L, or any combination thereof. In certain embodiments, the constant region comprises the sequence of SEQ ID NO: 11, and optionally further comprises the sequence of SEQ ID NO: 10.

In certain embodiments, the anti-CLDN18.2 antibody is humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536). In certain embodiments, the humanized light and heavy chains of the present disclosure are substantially non-immunogenic in humans and retain substantially the same affinity as or even higher affinity than the parent antibody to CLDN18.2.

In certain embodiments, the anti-CLDN18.2 antibody or an antigen-binding fragment thereof provided herein, comprises a heavy chain variable region comprising the sequence selected from the group consisting of SEQ ID NO: 12, SEQ ID NO: 13, SEQ ID NO: 14, and a homologous sequence thereof having at least 80% (e.g. at least 85%, 90%, 95%, 96%, 97%, 98%, or 99%) sequence identity yet retaining specific binding affinity to CLDN18.2, in particular human CLDN18.2.

In certain embodiments, the anti-CLDN18.2 antibody or an antigen-binding fragment thereof provided herein, antibody or an antigen-binding fragment thereof comprises a light chain variable region comprising the sequence selected from the group consisting of SEQ ID NO: 15, SEQ ID NO: 16, and a homologous sequence thereof having at least 80% (e.g. at least 85%, 90%, 95%, 96%, 97%, 98%, or 99%) sequence identity yet retaining specific binding affinity to CLDN18.2, in particular human CLDN18.2.

In certain embodiments, the anti-CLDN18.2 antibody comprises a heavy chain variable region and a light chain variable region, wherein: the heavy chain variable region comprises an amino acid sequence selected from the group consisting of SEQ ID NO: 12, SEQ ID NO: 13 and SEQ ID NO: 14, and the light chain variable region comprises an amino acid sequence selected from the group consisting of SEQ ID NO: 15 and SEQ ID NO: 16.

In certain embodiments, the humanized anti-CLDN18.2 antibodies may comprise the heavy chain variable region fused to the constant region of human IgG1 isotype and the light chain variable region fused to the constant region of human kappa chain.

The humanized anti-CLDN18.2 antibodies provided herein retained the specific binding affinity to a biological sample expressing CLDN18.2, and are at least comparable to, or even better than, the parent antibodies in that aspect. The humanized antibodies provided herein can also retain their functionality in that all antibodies can mediate cell killing by ADCC, CDC and induction of apoptosis induced by cross linking of the target at the tumor cell surface and direct inhibition of proliferation.

In certain embodiments, the anti-CLDN18.2 antibody is Hu18B10-HaLa. As used herein, the term “Hu18B10-HaLa” refers to a recombinant humanized IgG1 antibody specifically against human CLDN18.2 with high affinity and enhanced FcR engaging of NK cells, which can induce strong antibody-dependent cellular cytotoxicity (ADCC) activities. In certain embodiments, the anti-CLDN18.2 antibody comprises a heavy chain and a light chain, wherein the heavy chain comprises an amino acid sequence of SEQ ID NO: 17, and the light chain comprises an amino acid sequence of SEQ ID NO: 18.

The anti-CLDN18.2 antibodies used in the methods provided herein can also encompass various types of variants of the antibody sequences provided herein.

In certain embodiments, the variants comprise one or more modification(s) or substitution(s) in 1, 2, or 3 CDR sequences as mentioned above, in one or more FR sequences, in the heavy or light chain variable region sequences provided herein, and/or in the constant region (e.g., Fc region). Such antibody variants retain specific binding affinity to CLDN 18.2 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or increased effector function(s), improved FcRn receptor binding, increased pharmacokinetic half-life, pH sensitivity, and/or compatibility to conjugation (e.g., one or more introduced cysteine residues), to name a few. In certain embodiments, the anti-CLDN18.2 antibodies used in the methods provided herein also encompass a glycosylation variant having improved effector functions such as ADCC or CDC.

In certain embodiments, the anti-CLDN18.2 antibodies used in the methods provided herein is afucosylated. The term “afucosylation,” or “afucosylated,” refers to the reduced or eliminated core-fucose on the N-glycan attached to the antibody. The majority glycans of human IgG antibodies are known as G0, G1 and G2, which are complex biantennary molecules with core fucose residue carrying zero, one or two terminal galactose.

Afucosylated antibody variants can be made using methods known in the art, for example, as described in US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004).

In certain embodiments, the antibody glycosylation variant is afucosylated at Asn297 site of CH2 region in Fc of the antibody. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies.

In certain embodiments, the anti-CLDN18.2 antibodies used in the methods provided herein also encompass a cysteine-engineered variant, which comprises one or more introduced free cysteine amino acid residues. A free cysteine residue is one which is not part of a disulfide bridge. A cysteine-engineered variant is useful for conjugation with, for example a cytotoxic and/or imaging compound, a label, or a radioisoptype among others, at the site of the engineered cysteine, through for example a maleimide or haloacetyl. Methods for engineering antibodies or antigen-binding fragments to introduce free cysteine residues are known in the art, see, for example, WO2006/034488.

In certain embodiments, the constant region of the anti-CLDN 18.2 antibodies or antigen-binding fragments thereof used in the methods provided herein comprises one or more amino acid residue substitutions relative to SEQ ID NO: 9 (i.e. the wild-type sequence), selected from the group consisting of: L235V, F243L, R292P, Y300L, P396L, or any combination thereof. In certain embodiments, the constant region comprises the sequence of SEQ ID NO: 11.

In certain embodiments, the anti-CLDN18.2 antibodies used in the methods provided herein also encompass anti-CLDN18.2 antigen-binding fragments, such as a diabody, a Fab, a Fab′, a F(ab′)2, a Fd, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

In certain embodiments, the anti-CLDN18.2 antibodies used in the methods provided herein are bivalent, tetravalent, hexavalent, or multivalent. The term “valent” as used herein refers to the presence of a specified number of antigen binding sites in a given molecule. As such, the terms “bivalent”, “tetravalent”, and “hexavalent” denote the presence of two binding site, four binding sites, and six binding sites, respectively, in an antigen-binding molecule. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e. having the same sequences), or structurally different (i.e. having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope). The bi-specific antibodies that can be used in the methods provided herein can target both CLDN 18.2 and a checkpoint molecule, such as PD-1, PD-L1, PD-L2, CLTA-4, SIRPα TIM-3, LAG3, A2AR, CD160, 2B4, TGFβ, VISTA, BTLA, TIGIT, LAIR1, OX40, CD2, CD27, CD28, CD30, CD40, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-15, CD3, CD16 or CD83. Exemplary bi-specific antibodies that can be used in the methods provided herein include, without limitation, a bi-specific antibody targeting CLDN 18.2 and CD3, a bi-specific antibody targeting CLDN 18.2 and 4-1BB, a bi-specific antibody targeting CLDN 18.2 and TGFβ, a bi-specific antibody targeting CLDN 18.2 and SIRP, and a bi-specific antibody targeting CLDN 18.2 and IL-15.

The anti-CLDN18.2 antibodies used in the methods provided herein can also be antibody-drug conjugates (ADC) comprising any of the anti-CLDN18.2 antibodies mentioned above conjugated to a cytotoxic agent.

In certain embodiments, the cytotoxic agent can be any agent that is detrimental to cells or that can damage or kill cells. In certain embodiments, the cytotoxic agent is optionally a toxin, a chemotherapeutic agent (such as a DNA-alkylators, a topoisomerase inhibitor, a tubulin-binders, a growth inhibitory agent, or other anticancer drugs), or a radioactive isotope.

Examples of toxins include bacterial toxins and plant toxins, such as for example, diphtheria toxin, exotoxin A chain (from Pseudomonas aeruginosa), ricin, abrin, modeccin, alpha-sarcin, Aleurites fordii. proteins, dianthin proteins, Phytolaca americana proteins (PARI, PAPII, and PAP-S), Momordica charantia inhibitor, curcin, crotin, Sapaonaria officinalis inhibitor, gelonin, restrictocin, phenomycin, enomycin, and the tricothecenes (see, e.g., WO 93/21232). Such a large molecule toxin can be conjugated to the antibodies or antigen-binding fragments provided herein using methods known in the art, for example, as described in Vitetta et al (1987) Science, 238:1098.

The cytotoxic agent can also be small molecule toxins and chemotherapeutic agents, such as geldanamycin (Mandler et al (2000) Jour. of the Nat. Cancer Inst. 92(19):1573-1581; Mandler et al (2002) Bioconjugate Chem. 13:786-791), maytansine and maytansinoids (EP 1391213; Liu et al., (1996) Proc. Natl. Acad. Sci. USA 93:8618-8623; U.S. Pat. No. 5,208,020), calicheam icin (Lode et al (1998) Cancer Res. 58:2928; Hinman et al (1993) Cancer Res. 53:3336-3342), taxol, cytochalasin B, gramicidin D, ethidium bromide, emetine, mitomycin, etoposide, tenoposide, vincristine, vinblastine, vindesine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine), calicheamicin, maytansinoids, dolastatins, auristatins such as MMAE and MMAF (U.S. Pat. Nos. 5,635,483; 5,780,588), dolostatins, a trichothecene, exatecan, belotecan, deruxtecan, MMAE, MMAF, DM1, DM4, SN-38, PBD, eribulin and CC1065, and the derivatives thereof having cytotoxic activity.

The cytotoxic agent can also be a highly radioactive isotope. Examples include At211, I131, 1125, Y90, Re186, Sm153, Bi212, P32, Pb212 and radioactive isotopes of Lu. Methods of conjugation of a radioisotope to an antibody is known in the art, for example, via a suitable ligand reagent (see, e.g., WO94/11026; Current Protocols in Immunology, Volumes 1 and 2, Coligen et al, Ed. Wiley-Interscience, New York, N.Y., Pubs. (1991)). A ligand reagent has a chelating ligand that can bind, chelate or otherwise complex a radioisotope metal, and also has a functional group that is reactive with a thiol of cysteine of an antibody or antigen-binding fragment. Exemplary chelating ligands include DOTA, DOTP, DOTMA, DTPA and TETA (Macrocyclics, Dallas, Tex.).

In certain embodiments, in the ADC used in the methods provided herein, an antibody (or antigen-binding fragment thereof) is conjugated to one or more cytotoxic agents at an antibody: agent ratio of about 1 to about 20, about 1 to about 6, about 2 to about 6, about 3 to about 6, about 2 to about 5, about 2 to about 4, or about 3 to about 4.

c. Chemotherapy

In certain embodiments, the chemotherapy used in the methods provided herein comprises a combination of chemotherapeutic agents. The term “chemotherapeutic agent” is a biological (macromolecule) or chemical (small molecule) compound that can be used to treat cancer. The types of chemotherapeutic drugs include, but are not limited to, histone deacetylase inhibitor (HDACI), alkylating agents, antimetabolites, alkaloids, cytotoxic/anti-cancer antibiotics, topoisomerase inhibitors, tubulin inhibitors, proteins, antibodies, kinase inhibitors, and the like.

Examples of chemotherapeutic drugs include, erlotinib, afatinib, docetaxel, adriamycin, 5-FU (5-fluorouracil), panobinostat, gemcitabine, cisplatin, pemetrexed, carboplatin, paclitaxel, bevacizumab, trastuzumab, pertuzumab, metformin, temozolomide, tamoxifen, oteracil, doxorubicin, rapamycin, lapatinib, hydroxycamptothecin, trametinib, tegafur, gimeracil, leucovorin calcium (folinic acid) (LV), irinotecan hydrochloride (CPT-11), platinum (e.g., cisplatin), epirubicin, oxaliplatin, capecitabine.

In certain embodiments, the chemotherapy comprises a combination of chemotherapeutic agents selected from the group consisting of: LV, 5-FU, CPT-11, epirubicin, oxaliplatin, capecitabine, platinum (e.g., cisplatin), tegafur, gimeracil, oteracil, docetaxel, pemetrexed. In certain embodiments, the combination of chemotherapeutic agents comprises capecitabine and oxaliplatin. In certain embodiments, the combination of chemotherapeutic agents consists of capecitabine and oxaliplatin.

In certain embodiments, the chemotherapeutic regimen (or a combination of chemotherapeutic agents) may be selected from the group consisting of FOLFIRI chemotherapy, EOX chemotherapy, ECF chemotherapy, ECX chemotherapy, EOF chemotherapy, FLO chemotherapy, CAPOX chemotherapy, FOLFOX chemotherapy, DCF chemotherapy, SOX chemotherapy and FLOT chemotherapy. The drug combination used in FOLFIRI chemotherapy comprises or consists of: LV, 5-FU and CPT-l1. The drug combination used in EOX chemotherapy comprises or consists of: epirubicin, oxaliplatin and capecitabine. The drug combination used in ECF chemotherapy comprises or consists of: epirubicin, cisplatin and 5-FU. The drug combination used in ECX chemotherapy comprises or consists of: epirubicin, cisplatin and capecitabine. The drug combination used in EOF chemotherapy comprises or consists of: epirubicin, oxaliplatin and 5-FU. The drug combination used in FLO chemotherapy comprises or consists of: 5-FU, LV and oxaliplatin. The drug combination used in SOX chemotherapy comprises or consists of: tegafur, gimeracil, oteracil and oxaliplatin.

In certain embodiments, the chemotherapy comprises oxaliplatin of 20 mg/m² to 400 mg/m² (e.g., 40 mg/m², 60 mg/m², 80 mg/m², 100 mg/m², 120 mg/m², 130 mg/m², 140 mg/m², 160 mg/m², 180 mg/m², 200 mg/m², 220 mg/m², 240 mg/m², 300 mg/m², 340 mg/m², 380 mg/m² or 400 mg/m²). In certain embodiments, the chemotherapy comprises capecitabine of 500 mg/m² to 2000 mg/m² (e.g., 600 mg/m², 650 mg/m², 700 mg/m², 750 mg/m², 800 mg/m², 850 mg/m², 900 mg/m², 950 mg/m², 1000 mg/m², 1500 mg/m², 1700 mg/m² or 2000 mg/m²).

In certain embodiments, the chemotherapy used in the methods provided herein is the CAPOX regimen. In certain embodiments, the CAPOX regimen consists of Oxaliplatin 130 mg/m² administered intravenously on day 1 (e.g., no less than 2 hours of intravenous infusion, and a maximum of 8 cycles); and Capecitabine 850 mg/m2 or 1000 mg/m2 per dose by mouth twice daily (total dose 1700 or 2000 mg/m2 per day) on day 1 to day 14. There are several different CAPOX regimens that differ in the doses and ways in which the two drugs are given.

Other chemotherapeutic regimens mentioned above were described in PCT patent application PCT/JP2022/017017, which is herein incorporated by reference.

d. Immunotherapy

In certain embodiments, the immunotherapy used in the methods provided herein comprises an immunotherapeutic agent, such as a PD-1/PD-L1 axis inhibitor.

In certain embodiments, the PD-1/PD-L1 axis inhibitor is selected from the group consisting of: Nivolumab (OPDIVO; BMS-936558), Dostarlimab (TSR-042), Pembrolizumab (KEYTRUDA; MK-3475), MEDIO680 (AMP-514), MEDI4736, BI 754091, Pidilizumab (CT-011), Cemiplimab (LIBTAYO, REGN2810), Spartalizumab (PDR001), Cetrelimab (JNJ 63723283), Toripalimab (JS001), PF-06801591, Tislelizumab (BGB-A317), AMP-224 (GSK-2661380), ABBV-181, Lambrolizumab, Camrelizuma (SHR-1210), Sintilimab (Tyvyt, IB1308), Penpulimab(AK105), Zimberelimab, Retifanlimab, Serplulimab, Balstilimab,Geptanolimab, Prolgolimab, Ezabenlimab, Sasanlimab, Pimivalimab, Budigalimab, Nofazinlimab, Sindelizumab, MGA404, Sym021, BAT1306, HX008. Atezolizumab (TECENTRIQ; R05541267; MPDL3280A; RG7446), BMS-936559, Avelumab (bavencio), lodapolimab (LY3300054), Durvalumab (MEDI4736), CX-072 (Proclaim-CX-072), FAZ053, Envafolimab(KNO35), MDX-1105, STI-1040, CS1001, Adebrelimab (SHR-1316), SHR-1701, TOB2450, Bintrafusp, LP002, STI-3031, Cosibelimab, Pacmilimab, NM01, LDP, AMP-224, Garivulimab(BGB-A333), A167,SCD-135, Opucolimab, GR1405. In certain embodiments, the PD-1/PD-L1 axis inhibitor is Nivolumab.

In certain embodiments, the methods comprise administering to the subject a therapeutically effective amount of Hu18B10-HaLa, in combination with chemotherapy (e.g. CAPOX) and nivolumab.

In certain embodiments, the subject is determined to have PD-L1 expression in a cancer sample (e.g., in a tumor tissue sample).

In certain embodiments, the level of PD-L1 expression is measured by immuno-histochemistry (IHC) assay, hybridization assay or amplification assay.

In certain embodiments, the level of PD-L1 expression is quantified by membrane staining intensity measured by IHC using an anti-PD-L1 diagnostic reagent (e.g., anti-PD-L1 diagnostic antibody).

In certain embodiments, the PD-L1 expression is determined by IHC. Specific PD-L1 staining by IHC requires appropriate patients' materials, sensitive primary anti-PD-L1 antibodies, and suitable staining protocols. There are mainly three scoring algorithms for PD-L1 staining which are specific to tumor types and clinical decisions: the tumor proportion score (TPS), the immune proportion score (IPS) and the combined positivity score (CPS) (see, Schildhaus et al., Predictive value of PD-Li diagnostics. Pathologe. 2018 November; 39(6):498-519. doi: 10.1007/s00292-018-0507-x.).

TPS can be defined by the percentage of tumor cells positive for PD-L1 staining in relation to the total tumor cells in a biological sample, detailed description of which can be seen, for example, in Piper et al., Can PD-L1 tumor proportion score be used as the key to unlocking the KEYNOTE studies of pembrolizumab in advanced lung cancer? Transl Lung Cancer Res 2019; 8(5):715-722.

IPS can be defined by the percentage of tumor-associated immune cells positive for PD-L1 expression in relation to the total tumor-associated immune cells, detailed description of which can be seen, for example, in Yang et al., PD-L1 expression on tumor cells and tumor infiltrating immune cells in Chinese colorectal cancer patients. Journal of Clinical Oncology, Volume 38, Issue 15_suppl.

In certain embodiments, the level of PD-L1 expression is quantified by combined positive score (CPS), and wherein the CPS is determined by proportion of immune cells and tumor cells that are stained positive for PD-L1 relative to all viable tumor cells. CPS can be defined by the number of PD-L1 staining cells (including, without limitation, tumor cells, lymphocytes, macrophages) divided by the total number of viable tumor cells, multiplied by 100, detailed description can be seen, for example, in Yamashita et al., Prognostic impacts of the combined positive score and the tumor proportion score for programmed death ligand-1 expression by double immunohistochemical staining in patients with advanced gastric cancer. Gastric Cancer. 2020 January; 23(1):95-104; and Dako et al., Development of the combined positive score (CPS) for the evaluation of PD-L1 in solid tumors with the immunohistochemistry assay PD-L1 IHC 22C3 pharmDx. Journal of Clinical Oncology, Volume 35, Issue 15 suppl, 2017.

For example, the PD-L1 expression level of 5% (or CPS of 5) in a biological sample, such as a diseased tissue (e.g., cancerous tissue or tumor tissue), means that 5% of the cells (including, without limitation, tumor, lymphocytes and macrophages) in relation to the total tumor cells in the biological sample are positive for PD-L1 staining.

CPS for PD-L1 expression on a sample can be obtained using PD-L1 IHC 22C3 pharmDx or PD-L1 IHC 28-8 pharmDx. The PD-L1 IHC 28-8 pharmDx assay (Agilent Technologies, Santa Clara, California, USA), which is a kit for detecting PD-L1 expression, has been approved as a complementary diagnostic for use with nivolumab in cancers, such as non-squamous non-small cell lung cancer (NSQNSCL) and Squamous Cell Carcinoma of the Head and Neck (SCCHN). The PD-L1 IHC 28-8 pharmDx contains optimized reagents and protocol that are required to complete IHC staining of formalin-fixed, paraffin-embedded (FFPE) samples using Autostainer Link 48 and Dako PT Link Pre-treatment Module (Phillips T, Simmons P, Inzunza H D, et al. Development of an automated PD-L1 mmunohistochemistry (IHC) assay for non-small cell lung cancer. Appl Immuno Molec Morph 2015; 23(8):541-9.). In brief, the FFPE samples are first incubated with a primary monoclonal antibody to PD-L1 or the Negative Control Reagent (NCR), followed by incubation with a linker antibody specific to the host species of the primary antibody, and then a ready-to-use visualization reagent including secondary antibody molecules and horseradish peroxidase molecules coupled to a dextran polymer backbone, before being subject to light microscopy for visualization of PD-L1 staining.

The PD-L1 IHC 28-8 pharmDx was used in CheckMate-649, a randomized, multicenter, open-label trial in patients (n=1581) with previously untreated advanced or metastatic gastric cancer, gastroesophageal junction cancer, and esophageal adenocarcinoma. This trial enrolled patients regardless of PD-L1 expression level, and tumor samples from the patients were evaluated using the PD-L1 IHC 28-8 pharmDx assay at a central laboratory. This clinical trial demonstrated statistically significant improvement on overall survival (OS) and progression-free survival (PFS) for patients having tumors with PD-L1 expression of CPS≥5, which was not observed for patients having tumors with PD-L1 expression of CPS lower than 5 (information obtained from https://wwwfda.gov/media/124784/download).

In certain embodiments, the high level of PD-L1 expression corresponds to a CPS of above 5 (>5), optionally as measured by IHC assay using the PD-L1 diagnostic antibody (e.g. antibody 28-8), optionally on Dako autoscanner (e.g. Dako Autostainer Link 48 platform). In certain embodiments, the low level of PD-L1 expression corresponds to a CPS of no more than 5 (≤5), optionally as measured by IHC assay using the PD-L1 diagnostic antibody (e.g. antibody 28-8), optionally on Dako Autostainer Link 48 platform.

In certain embodiments, the anti-PD-L1 diagnostic antibody comprises the antibody selected from the group consisting of: 28-8, SP263, 22c3, and SP142.

In certain embodiments, the anti-PD-L1 diagnostic antibody 28-8 comprises a heavy chain variable domain comprising the amino acid sequence of SEQ ID NO: 30, and a light chain variable domain comprising the amino acid sequence of SEQ ID NO: 31.

In certain embodiments, the anti-PD-L1 diagnostic antibody SP263 comprises a heavy chain variable domain comprising the amino acid sequence of SEQ ID NO: 34, and a light chain variable domain comprising the amino acid sequence of SEQ ID NO: 35.

In certain embodiments, the anti-PD-L1 diagnostic antibody SP142 comprises a heavy chain variable domain comprising the amino acid sequence of SEQ ID NO: 32, and a light chain variable domain comprising the amino acid sequence of SEQ ID NO: 33.

In certain embodiments, the anti-PD-L1 diagnostic antibody 22C3 comprises a heavy chain variable domain comprising the amino acid sequence of SEQ ID NO: 36, and a light chain variable domain comprising the amino acid sequence of SEQ ID NO: 37.

In certain embodiments, the subject is determined to have in a cancer sample (e.g., in a tumor tissue sample): a) a high level of PD-L1 expression; or b) a low PD-L1 expression.

In certain embodiments, the subject is determined to have in the cancer sample: a) medium-to-high level of CLDN18.2 expression; or b) low level of CLDN18.2 expression. In certain embodiments, the subject is determined to have in the cancer sample: a) a high level of CLDN18.2 expression; b) a medium level of CLDN18.2 expression, or c) a low level of CLDN18.2 expression, as provided herein.

In certain embodiments, the subject is determined to have in a cancer sample (e.g., in a tumor tissue sample) at least 1% (e.g., at least 2%, at least 5% or at least 10%) of the cancer cells in the cancer sample having an expression level of CLDN18.2 characterized by membrane staining intensity of at least 1+, and less than 40% of the cancer cells in the cancer sample having an expression level of CLDN18.2 characterized by membrane staining intensity of at least 2+ (e.g. 2+ or 3+) as measured using an anti-CLDN18.2 diagnostic antibody (e.g. antibody 14G11), optionally on Leica autostainer (e.g. Leica Bond III LDT platform). In such embodiments, the subject is determined to have low level of PD-L1 expression that corresponds to a CPS of no more than 5 (≤5), or a high level of PD-L1 expression that corresponds to a CPS of above 5 (>5), optionally as measured by IHC assay using the PD-L1 diagnostic antibody (e.g. antibody 28-8).

In certain embodiments, the subject is determined to have in a cancer sample (e.g., in a tumor tissue sample) at least 40% of the cancer cells in the cancer sample (e.g., tumor tissue sample) having an expression level of CLDN18.2 characterized by membrane staining intensity of at least 2+ (e.g. 2+ or 3+), as measured using an anti-CLDN18.2 diagnostic antibody (e.g. antibody 14G11), optionally on Leica autostainer (e.g. Leica Bond III LDT platform). In such embodiments, the subject is determined to have low level of PD-L1 expression that corresponds to a CPS of no more than 5 (≤5), or a high level of PD-L1 expression that corresponds to a CPS of above 5 (>5), optionally as measured by IHC assay using the PD-L1 diagnostic antibody (e.g. antibody 28-8).

e. CLDN18.2-Expressing Cancer

“CLDN18.2-expressing cancer” as used herein refers any cancer or tumor involving cancer cells that express CLDN18.2 (e.g., low expression, medium expression, or high expression).

In certain embodiments, the CLDN18.2-expressing cancer is selected from the group consisting of gastric cancer, esophageal adenocarcinoma, gastric/gastro-esophageal junction (G/GEJ) cancer, lung cancer, bronchial cancer, bone cancer, liver and bile duct cancer, pancreatic cancer, breast cancer, liver cancer, ovarian cancer, testicle cancer, kidney cancer, bladder cancer, cholangiocarcinoma (biliary tract carcinoma), head and neck cancer, spine cancer, brain cancer, cervix cancer, uterine cancer, endometrial cancer, colon cancer, colorectal cancer, rectal cancer, anal cancer, esophageal cancer, gastrointestinal cancer, skin cancer, prostate cancer, pituitary cancer, stomach cancer, vagina cancer, thyroid cancer, glioblastoma, astrocytoma, melanoma, myelodysplastic syndrome, sarcoma, teratoma, and adenocarcinoma.

In certain embodiments, the CLDN18.2-expressing cancer is gastric cancer, esophageal adenocarcinoma, gastric/gastro-esophageal junction (G/GEJ) cancer, ovarian cancer, pancreatic cancer, cholangiocarcinoma (biliary tract carcinoma), colorectal cancer, or lung cancer.

In certain embodiments, the CLDN18.2-expressing cancer is HER2 negative.

In certain embodiments, the CLDN18.2-expressing cancer is gastric or gastroesophageal junction (mG/GEJ) adenocarcinoma, advanced gastric/gastro-esophageal junction cancer or esophageal adenocarcinoma.

In certain embodiments, the CLDN18.2-expressing cancer is locally advanced.

In certain embodiments, the CLDN18.2-expressing cancer is metastatic.

Although targeted therapy and immunotherapy have revolutionized systemic treatment of various cancers in the past decade, management of advanced and/or metastatic cancers is still a great challenge. “Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent. For example, many patients with advanced gastrointestinal cancers such as gastric cancer, pancreatic cancer, biliary tract carcinoma (BTC) (including gallbladder cancer and cholangiocarcinoma), or lung cancer still do not significantly benefit from current standard of care. Chemotherapy remains the mainstream treatment for most of these advanced stage cancer patients and their prognosis is still very poor.

An estimated of 55,440 people were diagnosed with pancreatic cancer and approximately 43,330 people died from the disease in US in year 2018. It is the fourth most common cause of cancer-related death in US. The prognosis of pancreatic cancer is extremely poor, with median survival time less than 6 months, and a 5-year survival rate (including localized type) is 9% (SEER).

Chemotherapy, including gemcitabine plus nab-paclitaxel or FOLFIRINOX, is still the 1st line standard of care.

Biliary tract carcinoma (BTC) (includes gallbladder cancer and Cholangiocarcinoma) is another tumor type with very poor prognosis. The median OS is 11.7 months and median PFS is 8.0 months when treated with Cisplatin plus gemcitabine as 1st line treatment in metastatic cholangiocarcinoma, which is still considered as one of preferred options of current standard of care (Valle J, 2010).

Lung cancer is the first estimated incidence and death worldwide in 2018 (Globocan, 2018). Although new targeted therapy (e.g., EGFR, ALK inhibitors, etc.) and immunotherapy had a significant progress these years and the overall survival in some subtypes has been significantly improved, many patients still do not benefit substantially, 5-year survival rate of patients with distant metastatic lung cancer (including NSCLC and SCLC) is only 5.2% (SEER).

Gastric cancer is the fifth most common and the third deadliest cancer worldwide based on GLOBOCAN 2018 data. Esophageal cancer is the 7th most frequently diagnosed cancer and 6th leading cause of cancer-related deaths in 2018 (Freddie et al., Global Cancer Statistics 2018: GLOBOCAN Estimates of Incidence and Mortality Worldwide for 36 Cancers in 185 Countries. CA CANCER J CLIN 2018; 68:394-424). The overall survival of gastroesophageal adenocarcinoma is 20-25%. Five-year survival rate of patients with metastatic gastric or gastroesophageal adenocarcinoma is only 5.3% (SEER). Trastuzumab is only benefit for patients with HER2 positive gastric or gastroesophageal cancer. Chemotherapy is still the mainstream for these patients.

In certain embodiments, the subject is treatment naïve. Treatment naïve refers to a condition or state of a patient who has not undergone any prior therapeutic intervention for the disease, in particular, for the CLDN18.2-expressing cancer. In other words, the treatment provided herein are provided to the subject as a first-line treatment.

In certain embodiments, the subject has not shown any resistance to a CLDN18.2 antagonist, a chemotherapy or a PD-1/PD-L1 axis inhibitor. The term “resistance” refers to the subject showing less responsive or refractory to the effects of a given treatment over time.

Resistance can be demonstrated by reduction in the treatment's ability to inhibit tumor growth, induce tumor cell death, or prevent tumor metastasis.

f. Pharmaceutical Compositions and Administration Routes

The CLDN18.2 antagonist, chemotherapeutic agent and immunotherapeutic agent described above may each be administered in the form of any suitable pharmaceutical composition. The term “pharmaceutical composition” refers a formulation comprising a therapeutically effective agent (e.g., the CLDN18.2 antagonist, chemotherapeutic agent, immunotherapeutic agent described above), preferably together with pharmaceutically acceptable carriers, diluents and/or excipients. The pharmaceutical composition is useful for treating, preventing, or reducing the severity of a disease or disorder by administration of said pharmaceutical composition to a subject.

Pharmaceutical compositions are usually provided in a uniform dosage form and may be prepared in a manner known in the art. A pharmaceutical composition may, for example, be in the liquid dosage form such as solution or suspension, or solid dosage forms such as tablets and capsules. The pharmaceutical compositions described herein are generally applied in a “therapeutically effective amount” and in a “pharmaceutically acceptable preparation”. The term “pharmaceutically acceptable”, as used herein, refers to the non-toxicity of a material that does not interact with the action of the active component of the pharmaceutical composition. For example, the term “pharmaceutically acceptable” may that the designated carrier, vehicle, diluent, excipient(s), and/or salt is generally chemically and/or physically compatible with the other ingredients comprising the formulation, and physiologically compatible with the recipient thereof.

The pharmaceutical compositions described herein may contain salts, buffers, preservatives, and optionally other therapeutic agents. In one embodiment, the pharmaceutical compositions of the present disclosure comprise one or more pharmaceutically acceptable carriers, diluents and/or excipients.

In certain embodiments, the administration is via oral, nasal, intravenous, subcutaneous, sublingual, or intramuscular administration.

In certain embodiments, the CLDN18.2 antagonist is administered at a dosage of about 1 mg/kg to about 30 mg/kg (e.g., 2 mg/kg, 4 mg/kg, 6 mg/kg, 8 mg/kg, 10 mg/kg, 12 mg/kg, 14 mg/kg, 16 mg/kg, 18 mg/kg, 20 mg/kg, 22 mg/kg, 24 mg/kg, 26 mg/kg, 28 mg/kg or 30 mg/kg).

In certain embodiments, the CLDN18.2 antagonist is administered at a dosage of about 4 mg/kg or 6 mg/kg.

In certain embodiments, the CLDN18.2 antagonist is administered at a dosing interval of once a week to once every 12-weeks (Q1W, Q2W, Q3W, Q4W, Q5W, Q6W, Q7W, Q8W, Q9W, Q10W, Q11W or Q12W), or once a month to once every 3 months (QIM, Q2M or Q3M), or once quarterly.

In certain embodiments, the CLDN18.2 antagonist is administered at a dosing interval of once every 2-weeks (Q2W) or once every 3-weeks (Q3W).

In certain embodiments, the CLDN18.2 antagonist is administered at a dosing of 4 mg/kg with interval of once every 2-weeks (Q2W) or administered at a dosing of 6 mg/kg with interval of once every 3-weeks (Q3W).

In certain embodiments, the administration of the CLDN18.2 antagonist is prior to, simultaneously with, or after the administration of the chemotherapy and/or the immunotherapy.

### III. Kit

In another aspect, the present disclosure also provides a kit useful in identifying and treating an individual having a CLDN18.2-expressing cancer who may benefit from a treatment, wherein the treatment comprises a CLDN18.2 antagonist and chemotherapy. In certain embodiments, the treatment further comprises an immunotherapy. In certain embodiments, the kit comprises an anti-CLDN18.2 diagnostic reagent (e.g., anti-CLDN18.2 diagnostic antibody) described herein and a package insert comprising instructions for using the treatment mentioned above in an individual having medium-to-high, or low level of the CLDN18.2 expression.

As used herein, the term “package insert” refers to instructions included in a commercial package of medicines that contain information about, for example, indications, dosage, usage, administration, contraindications, other medicines to be combined with the packaged product, and/or warnings concerning the use of such medicines. In certain embodiments, the instructions comprise selecting a subpopulation having low level of the CLDN18.2 protein for the treatment.

The kit may further comprise other materials desirable from a commercial and user standpoint, including other buffers, diluents, filters, needles, and syringes.

### Examples

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 1: Detection of Claudin18.2 Expression in Gastric/Gastroesophageal Junction Cancer Using Leica Bond III LDT Assay

The expression status of Claudin 18.2 was evaluated using an indirect immunohistochemical (IHC) technique with our internally developed and central-lab further validated mouse anti-human Claudin18.2 monoclonal antibody clone 14G11 on Leica auto-staining platform (Leica Bond III Automated IHC Stainer, Leica, Germany). Briefly, Immunohistochemistry (IHC) was performed on slides of 4% neutral buffered formalin fixed paraffin embedded samples. Sections were cut at a thickness of 4 μm and mounted onto positive charged slides. After deparaffinization and rehydration, all slides were proceeded to antigen retrieval by heating in BOND™ Epitope Retrieval Solution 2 (Leica, AR9640) for 30 minutes at 100° C., subsequently quenched, blocked with Peroxidase inhibitor and incubated with mouse anti-CLDN18.2 monoclonal antibody (clone 14G11) at dynamic range of concentration (0.45 pg/mL) diluted in primary antibody buffer (Leica Biosystem, AR9352) for 30 minutes. Antibody binding was amplified by post-primary IgG linker and visualized with BOND™ Polymer Detection (Leica, DS9800) by the formation of a brown precipitates. Sections were finally counterstained with Hematoxylin and mounted with permanent mounting medium.

IHC result was scored by board-certified pathologists under light field by microscope based on membrane staining intensity (neg (0), weak (1+), moderate (2+), strong (3+)) as well as Tumor Proportion Score (TPS) defined as the percentage of viable invasive tumor cells showing i 1+Claudin 18.2 staining intensity among all viable invasive tumor cells. Normal and/or adjacent uninvolved tissues are not included in the scoring assessment.

Below is the definition of claudin18.2 (CLDN 18.2) of H/M expression, Low expression and Rest expression.

H/M(high/medium expression): Patient samples showing ≥40% tumor cell with staining ≥2+ as high/medium expression group, in which ≥70% tumor cell with staining ≥2+ as high expression subgroup, or ≥40% tumor cell with staining ≥2+ and <70% tumor cell with staining 2 2+ as medium expression subgroup.

L (low expression): Patient samples showing ≥10% tumor cell with staining ≥1+ and <40% tumor cell with staining 2+ or 3+ as low expression group.

R (rest expression): Patient samples showing <10% tumor cell with staining ≥1+ as rest expression group.

### Example 2: Efficacy of Hu18B10-HaLa+CAPOX or Hu18B10-HaLa+CAPOX+Nivolumab in Claudin18.2 Expressing G/GEJ Cancer

### Hu18B10-HaLa+CAPOX (Cohort C):

A phase I/IIa clinical study was designed to evaluate the safety, tolerability, pharmacokinetics and preliminary efficacy of claudin18.2 monoclonal antibody Hu18b10-HaLa in the treatment of locally advanced unresectable or metastatic solid tumors.

Administration: Hu18B10-HaLa (6 mg/kg administrated IV on Day 1, Q3W) in combination with CAPOX (capecitabine 1000 mg/m2 twice daily on Days 1-14 of each 3-week cycle plus IV oxaliplatin 130 mg/m2 on Day 1 Q3W) for treating gastric/gastro-esophageal junction (G/GEJ) cancers. The result was shown in Table 1 with cutoff date in Apr. 18, 2024. The CLDN18.2 expression level was required to be (≥3 10% tumor cell with staining 1+); however, some patients treated earlier in the study at the same dose level didn't have their CLDN18.2 expression tested (reported as unknown). The expression status of claudin 18.2 was determined using an indirect immunohistochemical (IHC) technique with our internally developed and central-lab further validated mouse anti-human Claudin18.2 monoclonal antibody clone 14G11 on Leica auto-staining platform (Leica Bond III Automated IHC Stainer, Leica, Germany), see details in Example 1.

### Hu18B10-HaLa+CAPOX+Nivolumab (Cohort G):

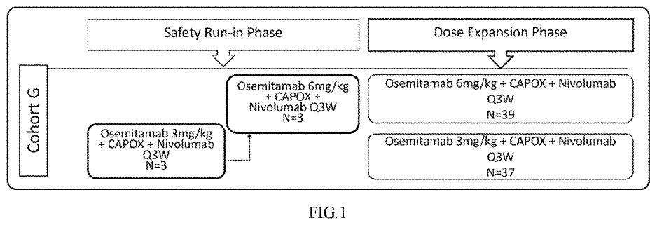

A phase I/IIa study was designed to evaluate combination of Hu18B10-HaLa (same as Osemitamab in FIG. 1), nivolumab and chemotherapy as first-line treatment in subjects with claudin18.2 positive locally advanced or metastatic gastric or gastroesophageal junction (gastric/GEJ) adenocarcinoma. Eligible patients include HER2 negative or unknown, unresectable locally advanced or metastatic G/GEJ cancer, and previously untreated with systemic anti-cancer therapies for advanced disease.

Administration: Hu18B10-HaLa (6 mg/kg or 3 mg/kg administrated IV on Day 1, Q3W) combine with CAPOX (capecitabine 1000 mg/m2 twice daily on Days 1-14 of each 3-week cycle plus IV oxaliplatin 130 mg/m2 on Day 1 Q3W), and combine with nivolumab (360 mg administrated IV on Day 1, Q3W; no more than 2 years), study design as FIG. 1. The result was shown in Table 1 with cutoff date in Apr. 18, 2024. The expression status of claudin 18.2 was determined using an indirect immunohistochemical (IHC) technique with our internally developed and central-lab further validated mouse anti-human Claudin18.2 monoclonal antibody clone 14G11 on Leica auto-staining platform (Leica Bond III Automated IHC Stainer, Leica, Germany), see details in Example 1.

The results are cross compared with other clinical studies (see details below in Comparison Study):
1) FAST, which is a randomised phase II study for Zolbetuximab+EOX (epirubicin, capecitabine and oxaliplatin) versus EOX alone for first-line treatment of advanced CLDN18.2-positive gastric and gastro-oesophageal adenocarcinoma;
2) GLOW, which is a Phase III study for Zolbetuximab+CAPOX in 1 L claudin-18.2+ (CLDN18.2+)/HER2− locally advanced (LA) or metastatic gastric or gastroesophageal junction (mG/GEJ) adenocarcinoma; and
3) CHECKMATE 649, which is an efficacy study of nivolumab plus chemotherapy against chemotherapy in stomach cancer or stomach/esophagus junction cancer.

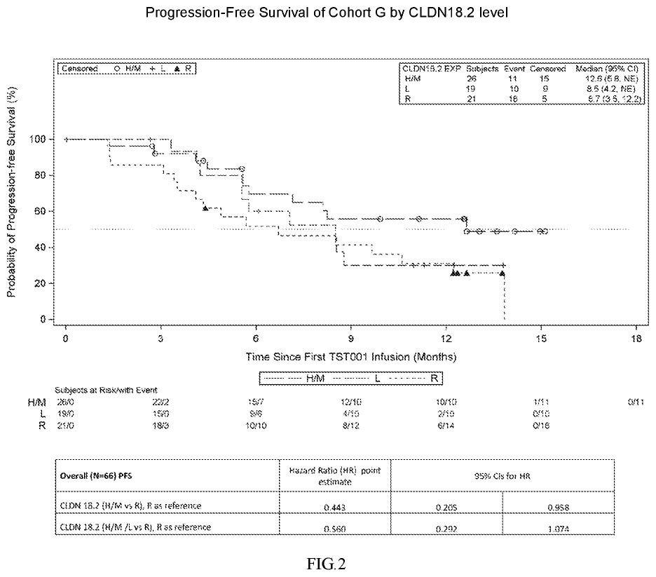

Table 1 and FIG. 2 shows that in comparison with Checkmate 649 study, in population with all PDL1, CPS, Hu18B10-hala in combination with Nivolumab and Oxaliplatin plus Capecitabine as first line treatment for First-line Advanced or Metastatic Gastric or Gastroesophageal Junction (Gastric/GEJ) Adenocarcinoma subjects has better outcomes in mPFS for CLDN 18.2 expression in H/M or L group with a cut-off date of Apr. 18, 2024. In particular, the mPFS of the H/M group is significantly longer than the mPFS of nivolumab plus chemotherapy arm from CheckMate 649 regardless of PD-L1 CPS status. Compared with Checkmate 649 study population with PDL1 CPS<5, the H/M group had better tumor responses. H/M and L group all showed better ORR than Checkmate 649 study population with PDL1 CPS<5.

For Efficacy analysis of Cohort G in patients (with known PD-L 1 CPS, CLDN 18.2), there is a clear trend between anti-tumor efficacy and CLDN18.2 expression, which leads to mPFS of 12.6 months for the patients with H/M expression, 8.5 months in L expression and 6.7 months in R Expression. The mPFS was 12.6 months in patients with H/M CLDN18.2 and PD-L1 CPS<5 as well. And the trend was also clearer in PD-L 1 CPS<5 subgroup. Besides, we compared the subsets by CLDN18.2 expression levels as an alternative approach. There was a clear trend between anti-tumor efficacy and CLDN18.2 expression which favored by HR (Hazard Ratio) of PFS with 0.56 (H/M/L vs R), and 0.443 (H/M vs R)) in patients (with known PD-L1 CPS, CLDN 18.2), and with 0.602 (H/M/L vs R), and 0.388 (H/M vs R)) in patients with PD-L1 CPS<5.

It is clear from the below Tables 2A and 2B that, Hu18B10-HaLa+CAPOX has efficacy outcomes, ORR, mPFS or mDOR that demonstrate better tumor responses than than the FAST study in all patients (CLDN18.2; 3 40% tumor cells staining 2+ or 3+) or in lower expressors only (CLDN18.2 expression ≥40-69% tumor cells staining 2+ or 3+), or than the GLOW study in patients (CLDN18.2 expression ≥75% tumor cell staining 2+ or 3+).

It is clear from the below Tables2A and 2B that, Hu18B10-HaLa+CAPOX+Nivolumab has efficacy outcomes, ORR or mPFS that demonstrate better tumor responses than Glow, Fast or Checkmate 649, particular in CLDN18.2 low expressors.

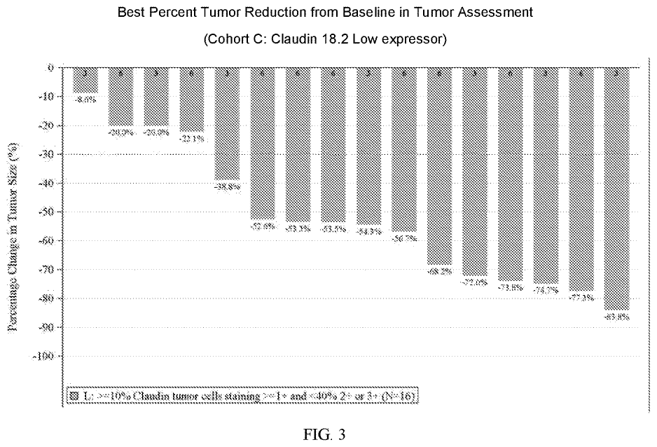

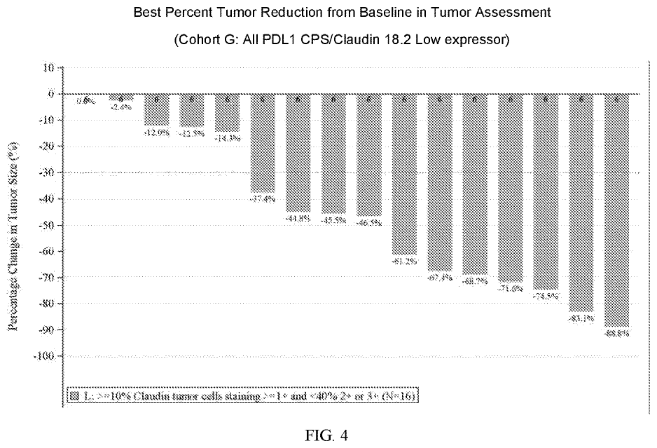

As shown in Table 1A and Table 1B, FIG. 3 (Cohort C) and FIG. 4, 5 (Cohort G), in low expressors (g 10% tumor cell with staining 1+, and ≤40% with staining 2+ or 3+), Hu18B10-HaLa plus CAPOX (further plus nivolumab) showed significant therapeutic efficacy.

TABLE 1

| | | | | | | | Checkmate 649 | |
|---|---|---|---|---|---|---|---|---|
| | Cohort G | | | | | | All PD-L1 | PD-L1 |
| | (TST001 + Nivolumab + CAPOX) | | | | | | CPS | CPS < 5 |
| | All patients (with PD-L1 | | | | | | (Nivolumab | (Nivolumab |
| | CPS, CLDN 18.2 known) | | | PD-L1 CPS < 5 | | | Plus | Plus |
| CLDN 18.2 | H/M | L | R | H/M | L | R | Chemotherapy) | Chemotherapy) |
| expression | (n = 26) | (n = 19) | (n = 21) | (n = 22) | (n = 16) | (n = 18) | NR | NR |
| mPFS | 12.6 | 8.5 | 6.7 | 12.6 | 7.1 | 5.7 | 7.7 | 7.5 |
| (months) | (95% CI | (95% CI | (95% CI | | | | | |
| | 5.8, NE) | 4.2, NE) | 3.5, 12.2) | | | | | |
| ORR | 68% | 61.1% | 50% | 71.4% | 60% | 47.1% | 58% | 55% |
| | (17/25) | (11/18) | (10/20) | (15/21) | (9/15) | (8/17) | | |

NR: not reported

TABLE 2A

| | | Cohort G * | |
|---|---|---|---|
| | | Hu18B10-HaLa + | Hu18B10-HaLa + |
| | Cohort C * | CAPOX + | CAPOX + |
| | Hu18B10-HaLa + | Nivolumab | Nivolumab |
| | CAPOX | All CPS | CPS > 5 |
| Claudin1 | Low expressor | Low expressor | Low expressor |
| 8.2 | N = 19 | N = 19 | N = 3 |
| Expression | (≥10% tumor cell | (≥10% tumor cell | (≥10% tumor cell |
| Level | with staining ≥1+, | with staining ≥1+, | with staining ≥1+, |
| | and <40% tumor cell | and <40% tumor | and <40% tumor cell |
| | with staining 2+ or | cell with staining | with staining 2+ or |
| | 3+ ) | 2+ or 3+) | 3+) |
| ORR | 57.9% (11/19) | 61.1% (11/18) | 66.7% (2/3) |
| mPFS | 12.7 months | 8.5 months | NE |
| | | (95% CI 4.2, NE) | (95% CI, 8.8 NE) |
| mDoR | 13.0 (5.5,NE) | 7.6months | NE |
| | | (95% CI 2.9, NE) | (95% CI 7.6, NE) |

TABLE 2B

| | GLOW | | FAST | | Checkmate 649 | |
|---|---|---|---|---|---|---|
| | Zolbetuximab + CAPOX | | Zolbetuximab + EOX | | nivolumab plus | |
| | ≥75% tumor | <75% tumor | All patients | Low expressor | chemotherapy | |
| Claudin18.2 | cell and | cell and | (≥40% tumor | (≥40-69% tumor | PD-L1 | PD-L1 |
| Expression | staining | staining | cells staining | cells and staining | All CPS | CPS > 5 |
| Level | 2+ or 3+ | 2+ or 3+ | 2+ or 3+) | 2+ or 3+) | NA | NA |
| ORR | 53.8% | NO data | Zolbetuximab + | NR | 58% | 60% |
| | (137/254) | (Not enroll | EOX vs EOX: | | | |
| | | this group) | 39% vs. 25% | | | |
| mPFS | 8.21 months | NO data | Zolbetuximab + | Zolbetuximab + | 7.7 months | 7.7 months |
| | | (Not enroll | EOX vs EOX: | EOX vs EOX: | | |
| | | this group) | 7.5 mo vs. 5.3 mo; | 4.3 mo vs 4.1 mo, | | |
| | | | HR 0.44; | HR 0.71, | | |
| | | | p < 0.0005 | p = 0.497 | | |
| mDoR | 6.28 months | NO data | NR | NR | 9.69 months | NR |
| | | (Not enroll | | | | |
| | | this group) | | | | |

*Cutoff date: Apr. 18, 2024 NR: not reported NE: Not reached

### Comparison Study: FAST and GLOW: Zolbetuximab+Chemotherapy

Zolbetuximab is a first-in-class, chimeric monoclonal antibody specific to a CLDN18.2 epitope. Early phase clinical studies (NCT00909025, NCT01197885, and NCT01671774) (Sahin U, 2015; Sahin U, 2018; Tdreci O, 2019) have shown that Zolbetuximab is well-tolerated with evidence of antitumor activity in patients with CLDN18.2 expressing solid tumors. In a phase IIa study when patient with advanced GC/GEJ cancer treated with Zolbetuximab monotherapy, in a subpopulation of patients with strong CLDN18.2 expression defined as ≥2+ staining intensity in 70% of tumor cells by immuno-histochemistry (IHC), the ORR was 14% (4/29) (Tiireci O, 2019, Ann Oncol. 2019 Sep. 1; 30(9):1487-1495. doi: 10.1093/annonc/mdz199.)

### FAST: Zolbetuximab+EOX (Epirubicin, Capecitabine and Oxaliplatin): A Randomised Phase II Study of Zolbetuximab Plus EOX Versus EOX Alone for First-Line Treatment of Advanced CLDN18.2-Positive Gastric and Gastro-Oesophageal Adenocarcinoma

FAST is a randomized phase 2 study (FAST; NCT01630083), which enrolled advanced gastric/gastro-oesophageal junction and oesophageal adenocarcinoma patients (aged ≥18 years) with moderate-to-strong CLDN18.2 expression in ≥40% tumor cells. Patients received first-line epirubicin+oxaliplatin+capecitabine (EOX, arm 1, n=84) every 3 weeks (Q3W), or zolbetuximab (800 mg/m2 then 600 mg/m2 Q3W)+EOX (arm 2, n=77). The primary endpoint was progression-free survival (PFS) and overall survival (OS) was a secondary endpoint.

Zolbetuximab in combination with EOX chemotherapy was shown to prolong overall and progression-free survival compared with EOX alone in patients with advanced GC/GEJ cancers that had moderate-to-strong CLDN18.2 expression (defined as membrane staining intensity of ≥2+ by IHC in ≥40% tumor cells). Median PFS was prolonged in Zolbetuximab+EOX arm versus EOX alone arm (7.5 mo vs. 5.3 mo; p<0.0005; HR 0.44; 95% CI 0.29, 0.67). Median OS (13.0 mo vs 8.3 mo; p<0.0005; HR 0.55; 95% CI 0.39, 0.77) were also significantly higher in Zolbetuximab+EOX arm versus EOX alone arm. Increased efficacy was more pronounced in patients with strong CLDN18.2 expression (≥2+ staining intensity in ≥70% tumor cells) and there was no clinically significant increase in PFS (4.3 mo vs 4.1 mo, HR 0.71; 95% CI 0.32, 1.57, p=0.497) and OS (8.3 mo vs 7.4 mo, HR 0.78; 95% CI 0.40, 1.49, p=0.401) in the lower expressors (40%-69% of tumor cells with moderate-to-strong CLDN18.2 staining). The lack of improved efficacy in the subgroups of the lower expressors led Astellas to run their pivotal phases 3 in “high expressors” defined as “moderate-to-strong membranous CLDN18 staining in ≥75% tumor cells”.

Two phase 3 were run by Astellas in 1 L claudin-18.2+ (CLDN18.2+)/HER2− locally advanced (LA) or metastatic gastric or gastroesophageal junction (mG/GEJ) adenocarcinoma evaluating the addition of Zolbetuximab to chemotherapy: SPOTLIGHT (which used a chemotherapy backbone of mFOLFOX6) and GLOW (backbone of CAPOX). Both studies were run with the same Claudin-18.2 selection criteria (moderate-to-strong membranous CLDN18 staining in 75% tumor cells) and led to similar conclusion of improved efficacy for the addition of Zolbetuximab. In the phase III study GLOW specifically, the ORR (95% CI) was 53.8% (46.58-60.99) in Zolbetuximab+CAPOX group, median PFS 8.21 months (95% CI, 7.46-8.84).

GLOW: Zolbetuximab+CAPOX (GLOW Phase III) Zolbetuximab+CAPOX in 1 L Claudin-18.2+ (CLDN18.2+)/HER2− Locally Advanced (LA) or Metastatic Gastric or Gastroesophageal Junction (mG/GEJ) Adenocarcinoma

Patients with CLDN18.2+ (moderate-to-strong membranous CLDN18 staining in ≥75% tumor cells by IHC)/HER2− LA unresectable or mG/GEJ adenocarcinoma were randomized 1:1 to zolbetuximab IV 800 mg/m2 (cycle 1, day [D] 1) followed by 600 mg/m2 (D1 in subsequent cycles)+CAPOX (oral capecitabine 1000 mg/m2 BID on D1-14 of each cycle; oxaliplatin IV 130 mg/m2 on D1 of each cycle) for eight 21-day cycles vs Placebo (PBO)+CAPOX; patients continued for ≥8 cycles with zolbetuximab or Placebo, plus capecitabine (investigator decision), until progression disease (PD) or a discontinuation criterium was met. The primary endpoint (EP) was PFS per RECIST v1.1 by IRC. OS was a key secondary EP; other secondary EPs included ORR and safety. Differences between treatment arms in PFS and OS were tested by stratified log-rank tests; OS was tested if PFS was significant.

The result shows that 507 patients were randomized 1:1 to zolbetuximab+CAPOX (N=254) or PBO+CAPOX (N=253). Both PFS (median 8.21 vs 6.80 mo, HR 0.687, P=0.0007) and OS (median 14.39 vs 12.16 mo, HR 0.771, P=0.0118) were significantly prolonged with zolbetuximab+CAPOX (Table 1); in patients with measurable disease, ORR (95% CI) was 53.8% (46.58-60.99) vs 48.8% (41.76-55.84) in zolbetuximab vs Placebo arm. The most common TEAEs with zolbetuximab+CAPOX were nausea (68.5% vs 50.2% in zolbetuximab vs Placebo arm), vomiting (66.1% vs 30.9%), and decreased appetite (41.3% vs 33.7%); serious TEAEs (47.2% vs 49.8%), grade ≥3 TEAEs (72.8% vs 69.9%), and drug-related TEAEs leading to death (2.4% vs 2.8%) were similar in both arms. (source: https://meetings.asco.org/abstracts-presentations/217906).

So, the data shown that Zolbetuximab have efficacy in claudin18.2 high cancer cell (expression defined as ≥2+ staining intensity in ≥70% of tumor cells by immuno-histochemistry (IHC)), or CLDN18 staining in ≥75% tumor cells, but not in medium and low Claudin18.2 expressors. There is a great unmet medical need to explore in claudin18.2 medium and low expression tumor cell which is not covered by Zolbetuximab. By default the standard of care for these patients remain chemotherapy or chemotherapy+a checkpoint inhibitor such as nivolumab.

### Comparison Study: Checkmate-649

Recently, immunotherapy is becoming another treatment option for gastric cancer patients. Nivolumab, one of the PD-I checkpoint inhibitors, has proved clinical benefit in multiple late-stage clinical trials. In a placebo-controlled phase 3 trial (ATTRACTION-2) in patients with unresectable advanced or recurrent gastric cancer that has progressed after two or more chemotherapy regimens, Nivolumab significantly improved overall survival (OS) (5.26 months vs. 4.14 moths; HR=0.63; P<0.001). The 12-month OS rate was 26.2% in nivolumab group (n=330) compared to 10.9% in the placebo group (n=163). ORR was 11% in nivolumab group. (Kang et al., Nivolumab inpatients with advanced gastric or gastro-oesophageal junction cancer refractory to, or intolerant of at least two previous chemotherapy regimens (ONO-4538-12, ATTRACTION-2): a randomised, double-blind, placebo-controlled, phase 3 trial. The Lancet, 390(10111), 2461-2471.)

Checkmate-649 is another phase 3 trial that compared nivolumab plus chemotherapy with chemotherapy alone in patients with untreated advanced G/GEJ cancer. More than 2,200 patients were randomized in three groups, 1581 patients were randomized to nivolumab (240 mg Q3W or 360 mg Q3W) plus chemotherapy (XELOX Q3W or FOLFOX Q2W) (n=789) or chemotherapy (n=792). Nivolumab plus chemotherapy significantly prolonged OS compared with chemotherapy in the PD-L1 CPS≥5 population (14.4 months vs. 11.1 months; HR=0.71; p<0.0001), Median progression-free survival (PFS) was 7.7 and 6.1 months, respectively (HR=0.68; P<0.0001), in the PD-L1 CPS≥5 population. However, patients with PD-L1 CPS<5 had minimal if any benefit from the addition of nivolumab: the OS of nivolumab plus chemotherapy was 12.4 months vs. 12.3 months, HR=0.94; and PFS was 7.5 months vs 8.2 months, HR=0.93.

Based on result of CheckMate-649, nivolumab in combination with chemotherapy is approved by FDA as the 1st line treatment for metastatic gastric cancer (regardless of PD-L1 expression), while the EMEA approved the combination regimen only for patients with PD-L1 CP≥5. NCCN guidelines and CSCO guidelines recommend nivolumab combined with chemotherapy for advanced metastatic gastric cancer patients with PD-L1 CPS≥5 (Category 1 evidence). For patients with advanced metastatic gastric cancer with PD-L1 CPS<5, nivolumab combined with chemotherapy can be used but is only Category 2 recommendation.

### CheckMate 649: Nivolumab Plus Chemotherapy: CheckMate 649: Efficacy Study of Nivolumab Plus Chemotherapy Against Chemotherapy in Stomach Cancer or Stomach/Esophagus Junction Cancer

In this multicenter, randomized, open-label, phase 3 trial (CheckMate 649), the study enrolled adults (≥18 years) with previously untreated, unresectable, non-HER2-positive gastric, gastro-esophageal junction, or esophageal adenocarcinoma, regardless of PD-ligand 1 (PD-L1) expression from 175 hospitals and cancer centers in 29 countries. Patients were randomly assigned (1:1:1 while all three groups were open) via interactive web response technology (block sizes of six) to nivolumab (360 mg every 3 weeks or 240 mg every 2 weeks) plus chemotherapy (capecitabine and oxaliplatin every 3 weeks or leucovorin, fluorouracil, and oxaliplatin every 2 weeks), or chemotherapy alone. Primary endpoints for nivolumab plus chemotherapy versus chemotherapy alone were OS or progression-free survival (PFS) by blinded independent central review, in patients whose tumors had a PD-L1 combined a positive score (CPS). Safety was assessed in all patients who received at least one dose of the assigned treatment.

### Example 3: Determination of CLDN18.2 and PD-L1 Co-Expression Status in Patient Tumor Samples

To investigate the expression level and overlapping status of CLDN18.2 and PD-L1 in GC/GEJ cancer, Immunohistochemistry (IHC) was performed on these 4% neutral buffered formalin fixed paraffin-embedded (FFPE) tumor sections using in-house developed and validated recombinant anti-CLDN18.2(14G11) and commercially available anti-PD-L1(28-8) monoclonal antibodies (e.g. Abcam, cat #ab205921). The sequences of the heavy chain and light chain variable regions of 28-8 monoclonal antibody are set forth in SEQ ID NOs. 30 and 31, respectively (see, U.S. Pat. No. 9,212,224B2). After deparaffinization and rehydration, all sections were proceeded to antigen retrieval by boiling in EnVision™ FLEX Target Retrieval Solution (Dako, K8002) for 25 minutes at 97-99° C., subsequently quenched, blocked with EnVision™ FLEX Peroxidase-Blocking Reagent (Dako, K8002) and incubated with appropriately diluted 14G11(0.6 ug/mL) and 28-8 (1 ug/mL) antibodies, respectively. Antibody binding was visualized with EnVision™ FLEX+, Mouse (LINKER), followed by EnVision™ FLEX/HRP and EnVision™ FLEX Substrate Working Solution (Dako, K8002). Sections were finally counterstained with Hematoxylin and mounted with permanent mounting medium.

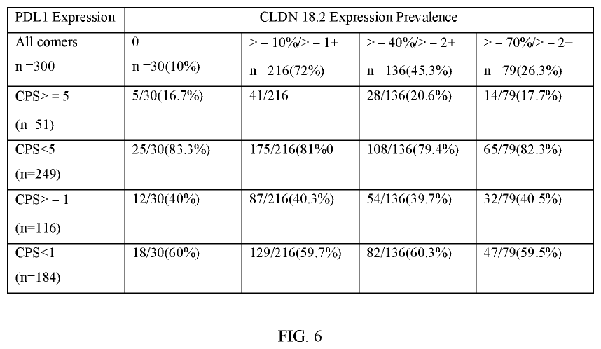

300 all corners were tested, the CLDN18.2 and PD-L1 expression levels and overlapping across GC/GEJ cancer tissues were summarized in FIG. 6. The result shows that most samples with CLDN18.2-expression in GC/GEJ Cancer have low PDL1 expression with CPS ≤5.

### Example 4: Evaluation of CLDN18.2 and PD-L1 Expression in Sections of GC-02-0007 PDX Tumor Model Using IHC Assay

### A. CLDN18.2 Expression Status in GC-02-0007 PDX Tumor Model

Immunohistochemistry (IHC) was performed on slides of 4% neutral buffered formalin fixed paraffin embedded PDX samples. Sections were cut at a thickness of 4-5 μm and mounted onto positive charged slides. After deparaffinization and rehydration, all slides were proceeded to antigen retrieval by heating in BOND Epitope Retrieval Solution 2 (Leica, AR9640) for 30 minutes at 100° C., subsequently quenched, blocked with Peroxidase inhibitor and incubated with mouse anti-claudin 18.2 monoclonal antibody (clone 14G11) at concentration of 0.15 pg/mL diluted in primary antibody buffer for 30 minutes. Antibody binding was amplified by post-primary IgG linker and visualized with BOND Polymer Detection (Leica, DS9800) by the formation of a brown precipitates. Sections were finally counterstained with Hematoxylin and mounted with permanent mounting medium.

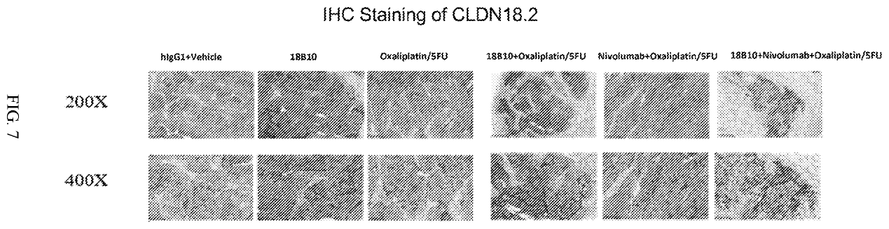

All viable tumor cells on the entire slide were evaluated and included in the scoring method. In general, at least 100 viable tumor cells were suggested for a percent score. IHC result was scored under light field by microscope based on membrane staining intensity (neg (0), weak (1+), moderate (2+), strong (3+)) as well as Tumor Proportion Score (TPS) defined as the percentage of viable invasive tumor cells showing ≥1+Claudin 18.2 staining intensity among all viable invasive tumor cells. Normal and/or adjacent uninvolved tissues are not included in the scoring assessment. The CLDN18.2 positivity was observed in GC-02-0007 PDX model with moderate to strong intensity at ≥75% tumor cells across all groups before and after treatment (see FIG. 7).

B. PD-L1 Expression Regulation Post Hu18B10-HaLa Alone or in Combination with Nivolumab/Chemotherapy Treatment in GC-02-0007 PDX Tumor Model

PD-L1 expression status was also assessed to better understand the immune modulation pre- and post Hu18B10-HaLa treatment in GC-02-0007 PDX tumor samples. Immunohistochemistry (IHC) was performed on these 4% neutral buffered formalin fixed paraffin-embedded (FFPE) tumor sections using commercially available Rabbit anti-human PD-L1 monoclonal antibodies (SP263). After deparaffinization and rehydration, all slides were proceeded to antigen retrieval in BOND Epitope Retrieval Solution 2 (Leica, AR9640) for 30 minutes at 97-99° C. Subsequently quenched, blocked with peroxidase inhibitor and incubated with appropriately diluted SP263 (0.2 ug/mL) antibodies for 30 minutes at room temperature (RT). Antibody binding was visualized with BOND Polymer Detection (Leica, DS9800) for 8 minutes on auto-staining Leica BOND III. Sections were finally counterstained with Hematoxylin and mounted with permanent mounting medium.

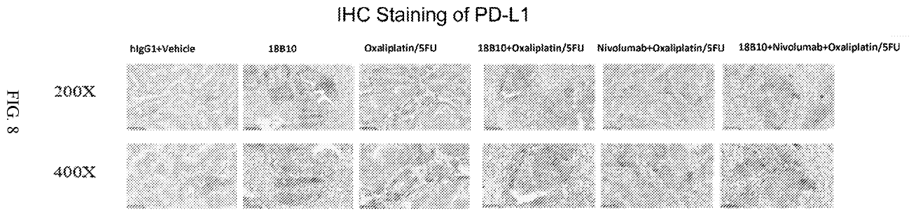

All samples were scored by combined positive score (CPS) of total stained immune and tumor cell relative to all viable tumor cell for PD-L 1 with membrane staining of different intensity (neg (0), weak (1+), moderate (2+), strong (3+)). Interestingly, the staining results displayed the treatment of Hu18B10-HaLa alone or in combination with Nivolumab/chemotherapy could upregulate the expression level of PD-L1 as compared with that of isotype control as depicted in FIG. 8 and Table 3.

TABLE 3
PD-L1 IHC result scoring on GC-02-0007 PDX tumor sections across groups

| Treatment | PD-L1IHC score |
|---|---|
| hIgG1 + Vehicle (isotype control) | 1+ |
| Hu18B10-HaLa | 2+ |
| Oxaliplatin/5FU | 2+ |
| Hu18B10-HaLa + Oxaliplatin/5FU | 3+ |
| Nivolumab + Oxaliplatin/5FU | 3+ |
| Hu18B10-HaLa + Nivolumab + Oxaliplatin/5FU | 3+ |

### Example 5. Efficacy of Hu18B10-HaLa in Combination with Nivolumab and Oxaliplatin Plus 5-FU on GC-02-0007 PDX Tumor Model on PBMC Humanized B-NDG-hIL-15 Mice

GC-02-0007 gastric cancer PDX was obtained from Beijing Cancer Hospital, passaged in NODSCID mice and PDX bank was established. As known in Example 4, the claudin18.2 expression is high which exceed 75% tumor cells in this mode, and the PD-L1 expression is low which CPS<5 in this mode. Each B-NDG-hIL-15 mouse was subcutaneously inoculated with a small tumor tissue block approximately 2-3 mm in diameter which sheared from a tumor decollement from a tumor bearing moue. 17 days after inoculation animals with tumor size at about 50-100 mm\hat{ }3 were selected and randomly divided into 6 groups each group consisting of 8 mice. Animals were intravenously inoculated with 5*10\hat{ }6 human PBMC in 0.1 mL. Animal were selected and dosed a few hours after human PBMC infusion. Animals from groups 1 to groups 6 were administered with 15 mg/kg hIgG1 and Vehicle, 10 mg/kg Hu18B10-HaLa, 1 mg/kg Oxaliplatin plus 10 mg/kg 5-FU, 10 mg/kg Hu18B1O-HaLa combination with 1 mg/kg Oxaliplatin plus 10 mg/kg 5-FU, 5 mg/kg Nivolumab combination with 1 mg/kg Oxaliplatin plus 10 mg/kg 5-FU, 10 mg/kg Hu18B1O-HaLa combination with 5 mg/kg Nivolumab and 1 mg/kg Oxaliplatin plus 10 mg/kg 5-FU, respectively. hIgG1, Hu18B10-HaLa and Nivolumab were administrated by intraperitoneal injection twice weekly for 3 weeks; Vehicle, Oxaliplatin and 5-FU were administrated by intravenous injection once. Animals were sacrificed at the end of the study with CO2 inhalation. Tumor size was measured twice a week in two dimensions using a caliper (INSIZE) and the volume was expressed in mm³ using the formula: V=0.5 a*b\hat{ }2 where a and b are the long and short diameters of the tumor, respectively. Results were analyzed using Prism GraphPad and expressed as mean=S.E.M. Comparisons between two groups were made by T-test. and the difference is considered significant if p is *≤0.05 and **≤0.01.

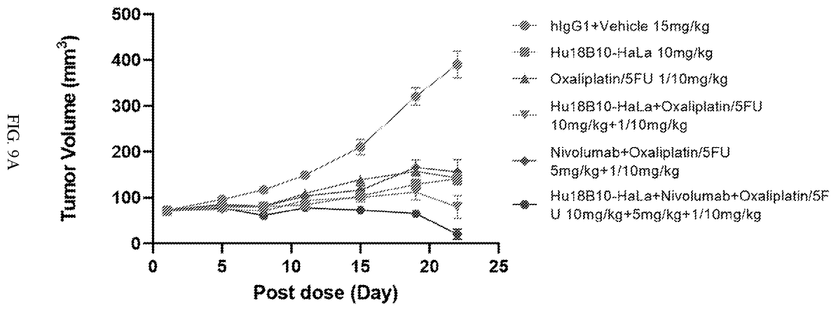

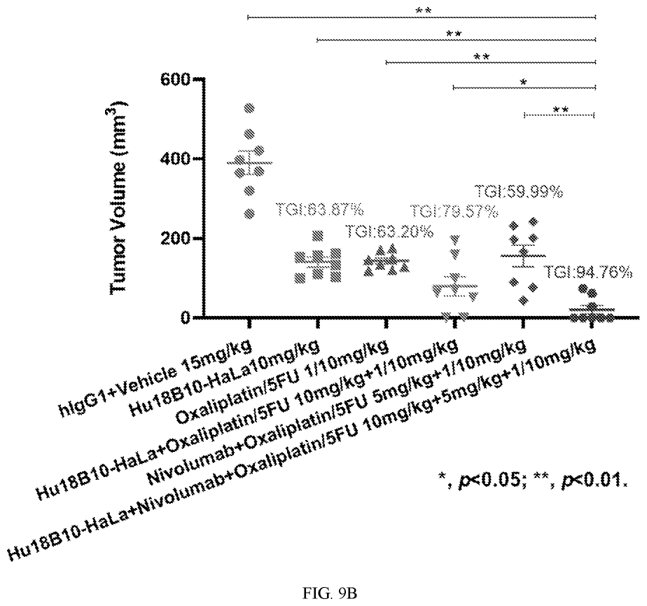

As shown in the FIGS. 9A and 9B, on human gastric cancer GC-02-0007 PDX tumor model with human CLDN18.2 high expression and human PD-L1 negative on B-NDG-hIL15 PBMC humanized mice, Hu18B10-HaLa in combination with Nivolumab and Oxaliplatin plus 5-FU was found better than any of the following groups: Oxaliplatin plus 5-FU alone, Hu18B10-HaLa in combination with Oxaliplatin plus 5-FU, and Nivolumab in combination with Oxaliplatin plus 5-FU in tumor growth inhibition.

TABLE 4
Amino acid sequences mentioned in the present disclosure

| SED ID NO. | Sequences | Region |
|---|---|---|
| 1 | GYNMN | 18B10_HCDR1 |
| 2 | NIDPYYGGTSYNQKFKG | 18B10_HCDR2 |
| 3 | MYHGNAFDY | 18B10_HCDR3 |
| 4 | KSSQSLLNSGNLKNYLT | 18B10_LCDR1 |
| 5 | WASTRKS | 18B10_LCDR2 |
| 6 | QNDYSYPLT | 18B10_LCDR3 |
| 7 | EFQLQQSGPELEKPGASVRISCKTSGYSFTGY | 18B10-VH |
| | NMNWVKQSNGESLEWIGNIDPYYGGTSYNQ | |
| | KFKGKATLTVDKSSSTAYMQLKSLTSEDSAV | |
| | YYCARMYHGNAFDYWGQGTTLTVSS | |
| 8 | DIVMTQSPSSLTVTAGEKVTMSCKSSQSLLNS | 18B10-VL |
| | GNLKNYLTWYQQKPGQPPKLLIYWASTRKSG | |
| | VPDRFTGSGSGTDFTLTLSSVQAEDLAVYYC | |
| | QNDYSYPLTFGAGTKLELK | |
| 9 | ASTKGPSVFPLAPSSKSTSGGTAALGCLVKDY | Human IgG1 |
| | FPEPVTVSWNSGALTSGVHTFPAVLQSSGLYS | heavy chain |
| | LSSVVTVPSSSLGTQTYICNVNHKPSNTKVDK | constant region |
| | KVEPKSCDKTHTCPPCPAPELLGGPSVFLFPPK | |
| | PKDTLMISRTPEVTCVVVDVSHEDPEVKFNW | |
| | YVDGVEVHNAKTKPREEQYNSTYRVVSVLT | |
| | VLHQDWLNGKEYKCKVSNKALPAPIEKTISK | |
| | AKGQPREPQVYTLPPSRDELTKNQVSLTCLV | |
| | KGFYPSDIAVEWESNGQPENNYKTTPPVLDS | |
| | DGSFFLYSKLTVDKSRWQQGNVFSCSVMHEA | |
| | LHNHYTQKSLSLSPGK | |
| 10 | RTVAAPSVFIFPPSDEQLKSGTASVVCLLNNF | Human Kappa |
| | YPREAKVQWKVDNALQSGNSQESVTEQDSK | light chain |
| | DSTYSLSSTLTLSKADYEKHKVYACEVTHQG | constant region |
| | LSSPVTKSFNRGEC | |
| 11 | ASTKGPSVFPLAPSSKSTSGGTAALGCLVKDY | Sequences of |
| | FPEPVTVSWNSGALTSGVHTFPAVLQSSGLYS | engineered Fc |
| | LSSVVTVPSSSLGTQTYICNVNHKPSNTKVDK | L235V/F243L/ |
| | KVEPKSCDKTHTCPPCPAPELVGGPSVFLLPP | R292P/Y300L/ |
| | KPKDTLMISRTPEVTCVVVDVSHEDPEVKFN | P396L |
| | WYVDGVEVHNAKTKPPEEQYNSTLRVVSVL | |
| | TVLHQDWLNGKEYKCKVSNKALPAPIEKTIS | |
| | KAKGQPREPQVYTLPPSRDELTKNQVSLTCL | |
| | VKGFYPSDIAVEWESNGQPENNYKTTPLVLD | |
| | SDGSFFLYSKLTVDKSRWQQGNVFSCSVMHE | |
| | ALHNHYTQKSLSLSPGK | |
| 12 | QVQLVQSGAEVKKPGASVKVSCKASGYTFTG | Hu18B10-Ha |
| | YNMNWVRQAPGQGLEWMGNIDPYYGGTSY | |
| | NQKFKGRVTMTIDKSTSTVYMELSSLRSEDT | |
| | AVYYCARMYHGNAFDYWGQGTTVTVSS | |
| 13 | QVQLVQSGAEVKKPGASVKVSCKASGYSFTG | Hu18B10-Hb |
| | YNMNWVRQAPGQGLEWMGNIDPYYGGTSY | |
| | NQKFKGRVTLTIDKSTSTVYMELSSLRSEDTA | |
| | VYYCARMYHGNAFDYWGQGTTVTVSS | |
| 14 | QVQLVQSGAEVKKPGASVKVSCKASGYSFTG | Hu18B10-Hc |
| | YNMNWVKQAPGQGLEWIGNIDPYYGGTSYN | |
| | QKFKGRVTLTIDKSTSTVYMELSSLRSEDTAV | |
| | YYCARMYHGNAFDYWGQGTTVTVSS | |
| 15 | DIVMTQSPDSLAVSLGERATINCKSSQSLLNS | Hu18B10_La |
| | GNLKNYLTWYQQKPGQPPKLLIYWASTRKSG | |
| | VPDRFSGSGSGTDFTLTISSLQAEDVAVYYCQ | |
| | NDYSYPLTFGGGTKVEIK | |
| 16 | DIVMTQSPDSLAVSLGERATMNCKSSQSLLNS | Hu18B10_Lb |
| | GNLKNYLTWYQQKPGQPPKLLIYWASTRKSG | |
| | VPDRFTGSGSGTDFTLTISSLQAEDVAVYYCQ | |
| | NDYSYPLTFGGGTKVEIK | |
| 17 | QVQLVQSGAEVKKPGASVKVSCKASGYTFTG | 18B10 HaLa |
| | YNMNWVRQAPGQGLEWMGNIDPYYGGTSY | Heavy chain |
| | NQKFKGRVTMTIDKSTSTVYMELSSLRSEDT | |
| | AVYYCARMYHGNAFDYWGQGTTVTVSSAST | |
| | KGPSVFPLAPSSKSTSGGTAALGCLVKDYFPE | |
| | PVTVSWNSGALTSGVHTFPAVLQSSGLYSLSS | |
| | VVTVPSSSLGTQTYICNVNHKPSNTKVDKKV | |
| | EPKSCDKTHTCPPCPAPELLGGPSVFLFPPKPK | |
| | DTLMISRTPEVTCVVVDVSHEDPEVKFNWYV | |
| | DGVEVHNAKTKPREEQYNSTYRVVSVLTVLH | |
| | QDWLNGKEYKCKVSNKALPAPIEKTISKAKG | |
| | QPREPQVYTLPPSRDELTKNQVSLTCLVKGFY | |
| | PSDIAVEWESNGQPENNYKTTPPVLDSDGSFF | |
| | LYSKLTVDKSRWQQGNVFSCSVMHEALHNH | |
| | YTQKSLSLSPGK | |
| 18 | DIVMTQSPDSLAVSLGERATINCKSSQSLLNS | 18B10 HaLa |
| | GNLKNYLTWYQQKPGQPPKLLIYWASTRKSG | Light chain |
| | VPDRFSGSGSGTDFTLTISSLQAEDVAVYYCQ | |
| | NDYSYPLTFGGGTKVEIK | |
| | RTVAAPSVFIFPPSDEQLKSGTASVVCLLNNF | |
| | YPREAKVQWKVDNALQSGNSQESVTEQDSK | |
| | DSTYSLSSTLTLSKADYEKHKVYACEVTHQG | |
| | LSSPVTKSFNRGEC | |
| 19 | QVQLQQSGPELVRPGASVKISCKASGYRFTRN | Heavy chain of |
| | YFHWVKQRPGQGLEWIGWIYPGGFDIEYSEK | recombinant |
| | FKGKATLTTDTSSSTAYMLLTSLTSEDSAVYY | anti-CLDN18.2 |
| | CAINYGSTFGYWGQGTLVTVSVAKTTPPSVY | (14G11) |
| | PLAPGSAAQTNSMVTLGCLVKGYFPEPVTVT | |
| | WNSGSLSSGVHTFPAVLQSDLYTLSSSVTVPS | |
| | STWPSQTVTCNVAHPASSTKVDKKIVPRDCG | |
| | CKPCICTVPEVSSVFIFPPKPKDVLTITLTPKVT | |
| | CVVVDISKDDPEVQFSWFVDDVEVHTAQTKP | |
| | REEQINSTFRSVSELPIMHQDWLNGKEFKCRV | |
| | NSAAFPAPIEKTISKTKGRPKAPQVYTIPPPKE | |
| | QMAKDKVSLTCMITNFFPEDITVEWQWNGQP | |
| | AENYKNTQPIMDTDGSYFVYSKLNVQKSNW | |
| | EAGNTFTCSVLHEGLHNHHTEKSLSHSPGK | |
| 20 | DVLMTQTPLSLPVSLGDQASISCRSSQNIVHS | Light chain of |
| | NGNTYLEWYLQRPGQSPKLLIYKVSNRFSGV | recombinant |
| | PDRFSGSGSGTDFTLKINRVEAEDLGVYYCFQ | anti-CLDN18.2 |
| | GSHVPFTFGSGTKLEIKRADAAPTVSIFPPSSE | (14G11) |
| | QLTSGGASVVCFLNNFYPKDINVKWKIDGSE | |
| | RQNGVLNSWTDQDSKDSTYSMSSTLTLTKDE | |
| | YERHNSYTCEATHKTSTSPIVKSFNRNEC | |
| 21 | QVQLQQSGPELVRPGASVKISCKASGYRFTRN | Heavy chain |
| | YFHWVKQRPGQGLEWIGWIYPGGFDIEYSEK | variable region |
| | FKGKATLTTDTSSSTAYMLLTSLTSEDSAVYY | of recombinant |
| | CAINYGSTFGYWGQGTLVTVSV | anti-CLDN18.2 |
| | | (14G11) |
| 22 | DVLMTQTPLSLPVSLGDQASISCRSSQNIVHS | Light chain |
| | NGNTYLEWYLQRPGQSPKLLIYKVSNRFSGV | variable region |
| | PDRFSGSGSGTDFTLKINRVEAEDLGVYYCFQ | of recombinant |
| | GSHVPFTFGSGTKLEIK | anti-CLDN18.2 |
| | | (14G11) |
| 23 | RNYFH | 14G11_HCDR1 |
| 24 | WIYPGGFDIEYSEKFKG | 14G11_HCDR2 |
| 25 | NYGSTFGY | 14G11_HCDR3 |
| 26 | RSSQNIVHSNGNTYLE | 14G11_LCDR1 |
| 27 | KVSNRFS | 14G11_LCDR2 |
| 28 | FQGSHVPFT | 14G11_LCDR3 |
| 29 | MAVTACQGLGFVVSLIGIAGIIAATCMDQWS | Amino acid |
| | TQDLYNNPVTAVFNYQGLWRSCVRESSGFTE | sequence of |
| | CRGYFTLLGLPAMLQAVRALMIVGIVLGAIGL | human |
| | LVSIFALKCIRIGSMEDSAKANMTLTSGIMFIV | CLDN18.2 |
| | SGLCAIAGVSVFANMLVTNFWMSTANMYTG | |
| | MGGMVQTVQTRYTFGAALFVGWVAGGLTLI | |
| | GGVMMCIACRGLAPEETNYKAVSYHASGHS | |
| | VAYKPGGFKASTGFGSNTKNKKIYDGGARTE | |
| | DEVQSYPSKHDYV | |
| 30 | METGLRWLLLVAVLKGVQCLSVEESGGRLV | Heavy chain |
| | TPGTPLTLTCTASGFTITNYHMFWVRQAPGK | variable region of |
| | GLEWIGVITSSGIGSSSTTYYATWAKGRFTISK | anti-PD-L1 |
| | TSTTVNLRITSPTTEDTATYFCARDY | diagnostic |
| | FTNTYYALDIWGPGTLVTVSS | antibody 28-8 |
| 31 | MDTRAPTQLLGLLLLWLPGARCALVMTQTPS | Light chain |
| | STSTAVGGTVTIKCQASQSISVYLAWYQQKP | variable region of |
| | GQPPKLLIYSASTLASGVPSRFKGSRSGTEYTL | anti-PD-L1 |
| | TISGVQREDAATYYCLGSAGS | diagnostic |
| | | antibody 28-8 |
| 32 | QSLEESGGRLVKPDETLTITCTVSGIDLSSNGL | Heavy chain |
| | TWVRQAPGEGLEWIGTINKDASAYYASWAK | variable region of |
| | GRLTISKPSSTKVDLKITSPTTEDTATYFCGRI | SP142 |
| | AFKTGTSIWGPGTLVTVSS | |
| 33 | AIVMTQTPSPVSAAVGGTVTINCQASESVYSN | Light chain |
| | NYLSWFQQKPGQPPKLLIYLASTLASGVPSRF | variable region of |
| | KGSGSGTQFTLTISGVQCDDAATYYCIGGKSS | SP142 |
| | STDGNAFGGGTEVVVR | |
| 34 | QSLEESGGRL VTPGTPLTLT CTASGFSLSN | Heavy chain |
| | HAISWVRQAP GKGLEWIGTINSDTHTYYAT | variable region of |
| | WPKGRFTISK TSSTTVDLKM TSPTTEDTAT | SP263 |
| | YFCARRIFSSSNIWGPGTLV TVSS | |
| 35 | AIVMTQTSSP VSAVVGGTVA | Light chain |
| | INCQASQSIYNNNWLSWFQQ | variable region of |
| | KPGQPPKLLIYLASTLASGV PSRFKGSGSG | SP263 |
| | TQFTLTISDV VCDDAATYYC | |
| | IGGESSNNDGIAFGGGTEVV VK | |
| 36 | DIVMSQSPSS LAVSAGEKVT MTCKSSQSLL | Heavy chain |
| | HTSTRKNYLA WYQQKPGQSPKLLIYWASTR | variable region of |
| | ESGVPDRFTG SGSGTDFTLT ISSVQAEDLA | 22C3 |
| | VYYCKQSYDVVTFGAGTKLE LK | |
| 37 | QVHLQQSGAE LAKPGASVKM SCKASGYTFT | light chain |
| | SYWIHWIKQR PGQGLEWIGYINPSSGYHEY | variable region of |
| | NQKFIDKATL TADRSSSTAY MHLTSLTSED | 22C3 |
| | SAVYYCARSGWLIHGDYYFD FWGQGTTLTV | |
| | SS | |

1. A method for treating a CLDN18.2-expressing cancer in a human subject in need thereof, comprising:
administering to the subject a therapeutically effective amount of a CLDN18.2 antagonist in combination with chemotherapy,
wherein the subject is determined to have medium-to-high, or low level of CLDN18.2 expression in a cancer sample (e.g., in a tumor tissue sample).

2. A method of identifying and treating a subject having a Claudin 18.2 (CLDN18.2)-expressing cancer who may benefit from a treatment comprising a CLDN18.2 antagonist in combination with chemotherapy, the method comprising:
(a) determining the level of CLDN18.2 protein in a cancer sample (e.g., in a tumor tissue sample), and
(b) selecting a subject having a medium-to-high level, or a low level of the CLDN18.2 protein for the treatment.

3. The method of claim 1 or 2, wherein the level of CLDN18.2 protein or the level of CLDN18.2 expression is measured by immune-histochemistry (IHC) assay, hybridization assay or amplification assay.

4. The method of any one of the preceding claims, wherein the level of CLDN18.2 protein or the level of CLDN18.2 expression is quantified by membrane staining intensity measured by IHC using an anti-CLDN18.2 diagnostic reagent (e.g., anti-CLDN18.2 diagnostic antibody).

5. The method of any one of the preceding claims, wherein the level of CLDN18.2 protein or the level of CLDN18.2 expression is quantified by percentage of cancer cells having a predetermined threshold of the membrane staining intensity measured by IHC using an anti-CLDN18.2 diagnostic reagent (e.g., anti-CLDN18.2 diagnostic antibody).

6. The method of any one of the preceding claims, wherein:
a) the medium-to-high level of CLDN18.2 protein or CLDN18.2 expression corresponds to at least 40% of the cancer cells in the cancer sample (e.g., tumor tissue sample) having an expression level of CLDN18.2 characterized by membrane staining intensity of at least 2+ (e.g. 2+ or 3+), as measured using an anti-CLDN18.2 diagnostic antibody optionally on Leica Bond III LDT platform, or corresponds to an equivalent readout as measured on another test platform, and/or
b) the low level of CLDN18.2 protein or CLDN18.2 expression corresponds to at least 1% (e.g., at least 2%, at least 5% or at least 10%) of the cancer cells in the cancer sample (e.g., tumor tissue sample) having an expression level of CLDN18.2 characterized by membrane staining intensity of at least 1+, and less than 40% of the cancer cells in the cancer sample (e.g., tumor tissue sample) having an expression level of CLDN18.2 characterized by membrane staining intensity of at least 2+ or 3+ as measured using an anti-CLDN18.2 diagnostic antibody optionally on Leica Bond III LDT platform, or corresponds to an equivalent readout as measured on another test platform.

7. The method of any one of the claims 4-6, wherein the anti-CLDN18.2 diagnostic antibody comprises HCDR1, HCDR2 and HCDR3 and LCDR1, LCDR2 and LCDR3 sequences, wherein:
the HCDR1 sequence comprises RNYFH (SEQ ID NO: 23), or a homologue sequence of at least 80% sequence identity thereof;
the HCDR2 sequence comprises WIYPGGFDIEYSEKFKG (SEQ ID NO: 24), or a homologue sequence of at least 80% sequence identity thereof;
the HCDR3 sequence comprises NYGSTFGY (SEQ ID NO: 25), or a homologue sequence of at least 80% sequence identity thereof;
the LCDR1 sequence comprises RSSQNIVHSNGNTYLE (SEQ ID NO: 26) or a homologue sequence of at least 80% sequence identity thereof;
the LCDR2 sequence comprises KVSNRFS (SEQ ID NO: 27) or a homologue sequence of at least 80% sequence identity thereof; and
the LCDR3 sequence comprises FQGSHVPFT (SEQ ID NO: 28) or a homologue sequence of at least 80% sequence identity thereof.

8. The method of claim 7, wherein the anti-CLDN18.2 diagnostic antibody comprises a VH and a VL, wherein:
a) the VH comprises an amino acid sequence of SEQ ID NO: 21 or a homologous sequence having at least 80% sequence identity thereof, and
b) the VL comprises an amino acid sequence of SEQ ID NO: 22 or a homologous sequence having at least 80% sequence identity thereof.

9. The method of any one of the claims 4-6, wherein the anti-CLDN18.2 diagnostic antibody comprises the antibody selected from the group consisting of: [EPR19202] (ab222512), 43-14A, SDT-102-24, MIL 93 and 3B10.

10. The method of any one of the claims 4-8, wherein
a) the medium-to-high level of CLDN18.2 protein or CLDN18.2 expression corresponds to at least 40% of the cancer cells in the cancer sample (e.g., tumor tissue sample) having an expression level of CLDN18.2 characterized by membrane staining intensity of at least 2+ (e.g. 2+ or 3+), as measured using 14G11 optionally on Leica Bond III LDT platform, or corresponds to an equivalent readout as measured on another test platform, and/or b) the low level of CLDN18.2 protein or CLDN18.2 expression corresponds to at least 1% (e.g., at least 2%, at least 5% or at least 10%) of the cancer cells in the cancer sample (e.g., tumor tissue sample) having an expression level of CLDN18.2 characterized by membrane staining intensity of at least 1+, and less than 40% of the cancer cells in the cancer sample (e.g., tumor tissue sample) having an expression level of CLDN18.2 characterized by membrane staining intensity of at least 2+ or 3+ as measured using 14G11 optionally on Leica Bond III LDT platform, or corresponds to an equivalent readout as measured on another test platform.

11. The method of any one of the preceding claims, wherein the anti-CLDN18.2 diagnostic antibody has a concentration of about 0.05-20 pg/mL (e.g., about 0.05-20 ug/ml, about 0.1-15 ug/ml, about 0.15-10 ug/ml, about 0.15-5 ug/ml, about 0.2-10 ug/ml, about 0.3-10 ug/ml, about 0.4-10 ug/ml, about 0.5-10 ug/ml, about 0.6-10 ug/ml, about 0.7-10 ug/ml, about 0.8-10 ug/ml, about 0.9-10 ug/ml, about 1-10 ug/ml or about 0.15-5 ug/ml).

12. The method of any one of the preceding claims, wherein the anti-CLDN18.2 diagnostic antibody is diluted in a dilution buffer for about 10-60 min (e.g., about 10-60 min, about 15-50 min, about 15-40 min, about 15-35 min, about 15-30 min, about 16-32 min or about 20-30 min).

13. The method of claim 12, wherein the dilution buffer is selected from the group consisting of Leica (AR9352), and PBS.

14. The method of any one of the preceding claims, wherein the IHC assay comprises antigen retrieval for about 15-120 min (e.g., about 15-120 min, about 25-100 min, about 35-90 min, about 45-80 min, about 55-90 min, about 65-80 min, about 70 min, about 15-30 min, about 20-30 min, or about 64-104 min).

15. The method of claim 14, wherein the antigen retrieval occurs at a temperature of about 50-100° C. (e.g., about 50° C.-100° C., about 60° C.-100° C., about 70° C.-100° C., about 80° C.-100° C., about 90° C.-100° C., about 92° C., about 94° C., about 96° C., about 97° C., about 98° C. or about 100° C.).

16. The method of any one of the preceding claims, wherein the chemotherapy comprises a chemotherapeutic agents selected from the group consisting of: erlotinib, afatinib, docetaxel, adriamycin, 5-fluorouracil (5-FU), panobinostat, gemcitabine, cisplatin, pemetrexed, carboplatin, paclitaxel, bevacizumab, trastuzumab, pertuzumab, metformin, temozolomide, tamoxifen, oteracil, doxorubicin, rapamycin, lapatinib, hydroxycamptothecin, trametinib, tegafur, gimeracil, leucovorin calcium (folinic acid) (LV), irinotecan hydrochloride (CPT-11), platinum (e.g., cisplatin), epirubicin, oxaliplatin, capecitabine.

17. The method of any one of the preceding claims, wherein the chemotherapy comprises a combination of chemotherapeutic agents.

18. The method of claim 17, wherein the chemotherapy comprises: 1) capecitabine and oxaliplatin (CAPOX); 2) LV, 5-FU and CPT-11 (FOLFIRI); 3) epirubicin, oxaliplatin and capecitabine (EOX); 4) epirubicin, cisplatin and 5-FU (ECF); 5) epirubicin, cisplatin and capecitabine (ECX); 6) epirubicin, oxaliplatin and 5-FU (EOF); 7) 5-FU, LV and oxaliplatin (FLO) or 8) tegafur, gimeracil, oteracil and oxaliplatin (SOX).

19. The method of any one of the preceding claims, wherein the treatment further comprises an immunotherapy, or wherein the method further comprises administering to the subject a therapeutically effective amount of an immunotherapy.

20. The method of any one of the preceding claims, wherein the immunotherapy comprises an immunotherapeutic agent, such as a PD-1/PD-L1 axis inhibitor.

21. The method of any one of preceding claims, wherein the subject is determined to have in a cancer sample (e.g., in a tumor tissue sample): a) a high level of PD-L1 expression; or b) a low level of PD-L1 expression.

22. The method of claim 21, wherein the level of PD-L1 expression is quantified by membrane staining intensity measured by IHC using an anti-PD-L1 diagnostic reagent (e.g., anti-PD-L1 diagnostic antibody).

23. The method of claim 22, wherein the level of PD-L1 expression is quantified by combined positive score (CPS), and wherein the CPS is determined by proportion of immune cells and tumor cells that are stained positive for PD-L1 relative to all viable tumor cells.

24. The method of claim 23, wherein the high level of PD-L1 expression corresponds to a CPS of above 5 (>5) as measured using antibody 28-8 by IHC, and/or the low level of PD-L1 expression corresponds to a CPS of no more than 5 (≤5) as measured using antibody 28-8 by IHC.

25. A method for treating a CLDN18.2-expressing cancer in a human subject in need thereof, comprising:
administering to the subject a therapeutically effective amount of a CLDN18.2 antagonist in combination with chemotherapy and a PD-1/PD-L1 axis inhibitor,
wherein the subject is determined to have low level of PD-L1 expression in a cancer sample (e.g., in a tumor tissue sample).

26. The method of claim 25, wherein the level of PD-L1 expression is measured by immuno-histochemistry (IHC) assay, hybridization assay or amplification assay.

27. The method of claim 26, wherein the level of PD-L1 expression is quantified by membrane staining intensity measured by IHC using an anti-PD-L1 diagnostic reagent (e.g., anti-PD-L1 diagnostic antibody).

28. The method of claim 27, wherein the level of PD-L1 expression is quantified by combined positive score (CPS), and wherein the CPS is determined by proportion of immune cells and tumor cells that are stained positive for PD-L1 relative to all viable tumor cells.

29. The method of claim 28, wherein the low level of PD-L1 expression corresponds to a CPS of no more than 5 (≤5) as measured using antibody 28-8 by IHC, optionally as measured by IHC assay on Dako Autostainer Link 48 platform.

30. The method of claim 27, wherein the anti-PD-L1 diagnostic antibody comprises the antibody selected from the group consisting of: 28-8, SP263, 22c3, and SP142.

31. The method of any one of claims 25-30, wherein the subject is determined to have in the cancer sample: a) medium-to-high level of CLDN18.2 expression; or b) low level of CLDN18.2 expression.

32. The method of any one of claims 25-31, wherein the level of CLDN18.2 expression is quantified as defined in any of claims 3-15.

33. The method of any one of claims 25-32, wherein the chemotherapy is as defined in any one of the claims 16-18.

34. The method of anyone of claims 25-33, wherein the PD-1/PD-L1 axis inhibitor is selected from the group consisting of: Nivolumab (OPDIVO; BMS-936558), Dostarlimab (TSR-042), Pembrolizumab (KEYTRUDA; MK-3475), MEDIO680 (AMP-514), MEDI4736, BI 754091, Pidilizumab (CT-011), Cemiplimab (LIBTAYO, REGN2810), Spartalizumab (PDR001), Cetrelimab (JNJ 63723283), Toripalimab (JS001), PF-06801591, Tislelizumab (BGB-A317), AMP-224 (GSK-2661380), ABBV-181, Lambrolizumab, Camrelizuma (SHR-1210), Sintilimab (Tyvyt, IBI308), Penpulimab(AK105), Zimberelimab, Retifanlimab, Serplulimab, Balstilimab,Geptanolimab, Prolgolimab, Ezabenlimab, Sasanlimab, Pimivalimab, Budigalimab, Nofazinlimab, Sindelizumab, MGA404, Sym021, BAT1306, HX008, Atezolizumab (TECENTRIQ; R05541267; MPDL3280A; RG7446), BMS-936559, Avelumab (bavencio), lodapolimab (LY3300054), Durvalumab (MEDI4736), CX-072 (Proclaim-CX-072), FAZ053, Envafolimab(KN035), MDX-1105, STI-1040, CS1001, Adebrelimab (SHR-1316), SHR-1701, TOB2450, Bintrafusp, LP002, STI-3031, Cosibelimab, Pacmilimab, NM01, LDP, AMP-224, Garivulimab(BGB-A333), A167,SCD-135, Opucolimab, GR1405.

35. The method of any one of the preceding claims, wherein the CLDN18.2 antagonist comprises an anti-CLDN18.2 antibody, such as a monoclonal anti-CLDN18.2 antibody, a bi-specific antibody targeting CLDN18.2 and a second antigen comprising an anti-CLDN18.2 antigen binding domain.

36. The method of any one of the preceding claims, wherein the CLDN18.2 antagonist comprises an anti-CLDN18.2 antigen binding domain comprising HCDR1, HCDR2 and HCDR3 and/or LCDR1, LCDR2 and LCDR3 sequences, wherein:
the HCDR1 sequence comprises GYNMN (SEQ ID NO: 1), or a homologue sequence of at least 80% sequence identity thereof;
the HCDR2 sequence comprises NIDPYYGGTSYNQKFKG (SEQ ID NO: 2),
or a homologue sequence of at least 80% sequence identity thereof;
the HCDR3 sequence comprises MYHGNAFDY (SEQ ID NO: 3), or a homologue sequence of at least 80% sequence identity thereof;
the LCDR1 sequence comprises KSSQSLLNSGNLKNYLT (SEQ ID NO: 4) or a homologue sequence of at least 80% sequence identity thereof;
the LCDR2 sequence comprises WASTRKS (SEQ ID NO: 5) or a homologue sequence of at least 80% sequence identity thereof; and
the LCDR3 sequence comprises QNDYSYPLT (SEQ ID NO: 6) or a homologue sequence of at least 80% sequence identity thereof.

37. The method of claim 36, wherein the CLDN18.2 antagonist comprises an anti-CLDN18.2 antigen binding domain comprising a VH and a VL, wherein:
the VH comprises an amino acid sequence of SEQ ID NO: 7 or a homologous sequence having at least 80% sequence identity thereof, and
the VL comprises an amino acid sequence of SEQ ID NO: 8 or a homologous sequence having at least 80% sequence identity thereof.

38. The method of any one of the preceding claims, wherein the anti-CLDN18.2 antagonist further comprises an immunoglobulin constant region, optionally a constant region of human Ig, or optionally a constant region of human IgG.

39. The method of claim 38, wherein the immunoglobulin constant region is a constant region of human IgG1, IgG2, IgG3, or IgG4.

40. The method of claim 39, wherein the immunoglobulin constant region is a constant region of human IgG1 comprising SEQ ID NO: 9, or a homologous sequence having at least 80% sequence identity thereof.

41. The method of any one of claims 38-40, wherein the immunoglobulin constant region comprises one or more amino acid residue substitutions or modifications conferring increased CDC or ADCC relative to wild-type constant region.

42. The method of claim 41, wherein the immunoglobulin constant region comprises one or more amino acid residue substitutions relative to SEQ ID NO: 9, selected from the group consisting of: L235V, F243L, R292P, Y300L, P396L, or any combination thereof.

43. The method of any one of claims 38-42, wherein the immunoglobulin constant region comprises the sequence of SEQ ID NO: 11.

44. The method of any one of claims 38-43, wherein the immunoglobulin constant region further comprises the sequence of SEQ ID NO: 10.

45. The method of any one of the preceding claims, wherein the CLDN18.2 antagonist is a humanized anti-CLDN18.2 antibody.

46. The method of claim 45, wherein the anti-CLDN18.2 antibody comprises a VH and a VL, wherein
the VH comprises an amino acid sequence selected from the group consisting of SEQ ID NO: 12, SEQ ID NO: 13 and SEQ ID NO: 14, and
the VL comprises an amino acid sequence selected from the group consisting of SEQ ID NO: 15 and SEQ ID NO: 16.

47. The method of claim 46, wherein the anti-CLDN18.2 antibody comprises a heavy chain and a light chain, wherein
the heavy chain comprises an amino acid sequence of SEQ ID NO: 17 or a homologous sequence having at least 80% sequence identity thereof, and
the light chain comprises an amino acid sequence of SEQ ID NO: 18 or a homologous sequence having at least 80% sequence identity thereof.

48. The method of any one of the preceding claims, wherein the CLDN18.2 antagonist is further linked to one or more conjugate moieties.

49. The method of claim 48, wherein the conjugate moiety comprises a clearance-modifying agent, a chemotherapeutic agent, a toxin(e.g., Exatecan, Belotecan, deruxtecan, MMAE, MMAF, DM1, DM4, SN-38, PBD, eribulin), a radioactive isotope, a lanthanide, a luminescent label, a fluorescent label, an enzyme-substrate label, a DNA-alkylators, a topoisomerase inhibitor, a tubulin-binders, a cytokine (e.g., IL-15, IL-2, IL-7), or other anticancer drugs.

50. The method of any one of the preceding claims, wherein the subject is human.

51. The method of any one of the preceding claims, wherein the administration is via oral, nasal, intravenous, subcutaneous, sublingual, or intramuscular administration.

52. The method of any one of the preceding claims, wherein the CLDN18.2 antagonist is administered at a dosage of about 1 mg/kg to about 30 mg/kg (e.g., 2 mg/kg, 4 mg/kg, 6 mg/kg, 8 mg/kg, 10 mg/kg, 12 mg/kg, 14 mg/kg, 16 mg/kg, 18 mg/kg, 20 mg/kg, 22 mg/kg, 24 mg/kg, 26 mg/kg, 28 mg/kg or 30 mg/kg).

53. The method of any one of the preceding claims, wherein the CLDN18.2 antagonist is administered at a dosage of about 4 mg/kg or 6 mg/kg.

54. The method of any one of the preceding claims, wherein the CLDN18.2 antagonist is administered at a dosing interval of once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

55. The method of any one of the preceding claims, wherein the CLDN18.2 antagonist is administered at a dosing interval of once every 2-weeks (Q2W) or once every 3-weeks (Q3W).

56. The method of any one of the preceding claims, wherein the CLDN18.2 antagonist is administered at a dosing of 4 mg/kg with interval of once every 2-weeks (Q2W) or administered at a dosing of 6 mg/kg with interval of once every 3-weeks (Q3W).

57. The method of any one of the preceding claims, wherein the administration of the CLDN18.2 antagonist is prior to, simultaneously with, or after the administration of the chemotherapy.

58. The method of any one of the preceding claims, wherein the CLDN18.2-expressing cancer is selected from the group consisting of gastric cancer, esophageal adenocarcinoma, gastric/gastro-esophageal junction (G/GEJ) cancer, lung cancer, bronchial cancer, bone cancer, liver and bile duct cancer, pancreatic cancer, breast cancer, liver cancer, ovarian cancer, testicle cancer, kidney cancer, bladder cancer, cholangiocarcinoma (biliary tract carcinoma), head and neck cancer, spine cancer, brain cancer, cervix cancer, uterine cancer, endometrial cancer, colon cancer, colorectal cancer, rectal cancer, anal cancer, esophageal cancer, gastrointestinal cancer, skin cancer, prostate cancer, pituitary cancer, stomach cancer, vagina cancer, thyroid cancer, glioblastoma, astrocytoma, melanoma, myelodysplastic syndrome, sarcoma, teratoma, and adenocarcinoma.

59. The method of claim 58, wherein the CLDN18.2-expressing cancer is gastric cancer, esophageal adenocarcinoma, gastric/gastro-esophageal junction (G/GEJ) cancer, ovarian cancer, pancreatic cancer, cholangiocarcinoma (biliary tract carcinoma), colorectal cancer, or lung cancer.

60. The method of claim 59, wherein the CLDN18.2-expressing cancer is gastric or gastroesophageal junction (mG/GEJ) adenocarcinoma, advanced gastric/gastro-esophageal junction cancer or esophageal adenocarcinoma.

61. The method of any one of the preceding claims, wherein the CLDN18.2-expressing cancer is HER2 negative.

62. The method of any one of the preceding claims, wherein the CLDN18.2-expressing cancer is locally advanced.

63. The method of any one of the preceding claims, wherein the CLDN18.2-expressing cancer is metastatic.

64. The method of any one of the preceding claims, wherein the subject is treatment naïve.

65. The method of any one of the preceding claims, wherein the subject has not shown any resistance to a CLDN18.2 antagonist, a chemotherapy or a PD-1/PD-L1 axis inhibitor.

66. The method of any one of the preceding claims, wherein the subject has a survival of a longer period as compared to a subject who has been treated with a reference antibody (e.g., Nivolumab or Zolbetuximab) in combination with chemotherapy.

67. The method of claim 66, wherein the subject has a survival of at least 5 months (e.g., at least 6 months, at least 7 months, at least 8 months, at least 9 months, at least 10 months, at least 11 months, at least 12 months, at least 13 months, at least 14 months or at least 15 months).

68. Use of a CLDN18.2 antagonist in the manufacture of a medicament for treating a CLDN18.2-expressing cancer in a subject in need thereof, wherein the treatment comprises administering to the subject the medicament in combination with chemotherapy, wherein the subject is determined to have medium-to-high, or low level of CLDN18.2 expression in a cancer sample (e.g., in a tumor tissue sample).

69. Use of a CLDN18.2 antagonist in the manufacture of a medicament for treating a CLDN18.2-expressing cancer in a subject in need thereof, wherein the medicament further comprises chemotherapy, wherein the subject is determined to have medium-to-high, or low level of CLDN18.2 expression in a cancer sample (e.g., in a tumor tissue sample).

70. A kit for use in identifying and treating a subject having a CLDN18.2-expressing cancer who may benefit from a treatment comprising a CLDN18.2 antagonist in combination with chemotherapy, comprising an anti-CLDN18.2 diagnostic reagent (e.g., anti-CLDN18.2 diagnostic antibody) and a package insert comprising instructions for using the treatment in a subject having medium-to-high, or low level of the CLDN18.2 expression.


SEQUENCE LISTING (37 sequences)

SEQ ID NO: 1 - protein, 5 aa, synthetic construct
  REGION 1..5: note = Synthetic
GYNMN

SEQ ID NO: 2 - protein, 17 aa, synthetic construct
  REGION 1..17: note = Synthetic
NIDPYYGGTSYNQKFKG

SEQ ID NO: 3 - protein, 9 aa, synthetic construct
  REGION 1..9: note = Synthetic
MYHGNAFDY

SEQ ID NO: 4 - protein, 17 aa, synthetic construct
  REGION 1..17: note = Synthetic
KSSQSLLNSGNLKNYLT

SEQ ID NO: 5 - protein, 7 aa, synthetic construct
  REGION 1..7: note = Synthetic
WASTRKS

SEQ ID NO: 6 - protein, 9 aa, synthetic construct
  REGION 1..9: note = Synthetic
QNDYSYPLT

SEQ ID NO: 7 - protein, 118 aa, synthetic construct
  REGION 1..118: note = Synthetic
EFQLQQSGPELEKPGASVRISCKTSGYSFTGYNMNWVKQSNGESLEWIGNIDPYYGGTSYNQKFKGKATLTVDKSSSTAYMQLKSLTSEDSAVYYCARMYHGNAFDYWGQGTTLTVSS

SEQ ID NO: 8 - protein, 113 aa, synthetic construct
  REGION 1..113: note = Synthetic
DIVMTQSPSSLTVTAGEKVTMSCKSSQSLLNSGNLKNYLTWYQQKPGQPPKLLIYWASTRKSGVPDRFTGSGSGTDFTLTLSSVQAEDLAVYYCQNDYSYPLTFGAGTKLELK

SEQ ID NO: 9 - protein, 330 aa, synthetic construct [sequence omitted]

SEQ ID NO: 10 - protein, 107 aa, synthetic construct [sequence omitted]

SEQ ID NO: 11 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 12 - protein, 118 aa, synthetic construct
  REGION 1..118: note = Synthetic
QVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSS

SEQ ID NO: 13 - protein, 118 aa, synthetic construct
  REGION 1..118: note = Synthetic
QVQLVQSGAEVKKPGASVKVSCKASGYSFTGYNMNWVRQAPGQGLEWMGNIDPYYGGTSYNQKFKGRVTLTIDKSTSTVYMELSSLRSEDTAVYYCARMYHGNAFDYWGQGTTVTVSS

SEQ ID NO: 14 - protein, 118 aa, synthetic construct
  REGION 1..118: note = Synthetic
QVQLVQSGAEVKKPGASVKVSCKASGYSFTGYNMNWVKQAPGQGLEWIGNIDPYYGGTSYNQKFKGRVTLTIDKSTSTVYMELSSLRSEDTAVYYCARMYHGNAFDYWGQGTTVTVSS

SEQ ID NO: 15 - protein, 113 aa, synthetic construct
  REGION 1..113: note = Synthetic
DIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIK

SEQ ID NO: 16 - protein, 113 aa, synthetic construct
  REGION 1..113: note = Synthetic
DIVMTQSPDSLAVSLGERATMNCKSSQSLLNSGNLKNYLTWYQQKPGQPPKLLIYWASTRKSGVPDRFTGSGSGTDFTLTISSLQAEDVAVYYCQNDYSYPLTFGGGTKVEIK

SEQ ID NO: 17 - protein, 448 aa, synthetic construct
  REGION 1..448: note = Synthetic
QVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 220 aa, synthetic construct
  REGION 1..220: note = Synthetic
DIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 441 aa, synthetic construct
  REGION 1..441: note = Synthetic
QVQLQQSGPELVRPGASVKISCKASGYRFTRNYFHWVKQRPGQGLEWIGWIYPGGFDIEYSEKFKGKATLTTDTSSSTAYMLLTSLTSEDSAVYYCAINYGSTFGYWGQGTLVTVSVAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 20 - protein, 219 aa, synthetic construct
  REGION 1..219: note = Synthetic
DVLMTQTPLSLPVSLGDQASISCRSSQNIVHSNGNTYLEWYLQRPGQSPKLLIYKVSNRFSGVPDRFSGSGSGTDFTLKINRVEAEDLGVYYCFQGSHVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 117 aa, synthetic construct
  REGION 1..117: note = Synthetic
QVQLQQSGPELVRPGASVKISCKASGYRFTRNYFHWVKQRPGQGLEWIGWIYPGGFDIEYSEKFKGKATLTTDTSSSTAYMLLTSLTSEDSAVYYCAINYGSTFGYWGQGTLVTVSV

SEQ ID NO: 22 - protein, 112 aa, synthetic construct
  REGION 1..112: note = Synthetic
DVLMTQTPLSLPVSLGDQASISCRSSQNIVHSNGNTYLEWYLQRPGQSPKLLIYKVSNRFSGVPDRFSGSGSGTDFTLKINRVEAEDLGVYYCFQGSHVPFTFGSGTKLEIK

SEQ ID NO: 23 - protein, 5 aa, synthetic construct
  REGION 1..5: note = Synthetic
RNYFH

SEQ ID NO: 24 - protein, 17 aa, synthetic construct
  REGION 1..17: note = Synthetic
WIYPGGFDIEYSEKFKG

SEQ ID NO: 25 - protein, 8 aa, synthetic construct
  REGION 1..8: note = Synthetic
NYGSTFGY

SEQ ID NO: 26 - protein, 16 aa, synthetic construct
  REGION 1..16: note = Synthetic
RSSQNIVHSNGNTYLE

SEQ ID NO: 27 - protein, 7 aa, synthetic construct
  REGION 1..7: note = Synthetic
KVSNRFS

SEQ ID NO: 28 - protein, 9 aa, synthetic construct
  REGION 1..9: note = Synthetic
FQGSHVPFT

SEQ ID NO: 29 - protein, 261 aa, synthetic construct
  REGION 1..261: note = Synthetic
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 30 - protein, 141 aa, synthetic construct
  REGION 1..141: note = Synthetic
METGLRWLLLVAVLKGVQCLSVEESGGRLVTPGTPLTLTCTASGFTITNYHMFWVRQAPGKGLEWIGVITSSGIGSSSTTYYATWAKGRFTISKTSTTVNLRITSPTTEDTATYFCARDYFTNTYYALDIWGPGTLVTVSS

SEQ ID NO: 31 - protein, 116 aa, synthetic construct
  REGION 1..116: note = Synthetic
MDTRAPTQLLGLLLLWLPGARCALVMTQTPSSTSTAVGGTVTIKCQASQSISVYLAWYQQKPGQPPKLLIYSASTLASGVPSRFKGSRSGTEYTLTISGVQREDAATYYCLGSAGS

SEQ ID NO: 32 - protein, 115 aa, synthetic construct
  REGION 1..115: note = Synthetic
QSLEESGGRLVKPDETLTITCTVSGIDLSSNGLTWVRQAPGEGLEWIGTINKDASAYYASWAKGRLTISKPSSTKVDLKITSPTTEDTATYFCGRIAFKTGTSIWGPGTLVTVSS

SEQ ID NO: 33 - protein, 112 aa, synthetic construct
  REGION 1..112: note = Synthetic
AIVMTQTPSPVSAAVGGTVTINCQASESVYSNNYLSWFQQKPGQPPKLLIYLASTLASGVPSRFKGSGSGTQFTLTISGVQCDDAATYYCIGGKSSSTDGNAFGGGTEVVVR

SEQ ID NO: 34 - protein, 114 aa, synthetic construct
  REGION 1..114: note = Synthetic
QSLEESGGRLVTPGTPLTLTCTASGFSLSNHAISWVRQAPGKGLEWIGTINSDTHTYYATWPKGRFTISKTSSTTVDLKMTSPTTEDTATYFCARRIFSSSNIWGPGTLVTVSS

SEQ ID NO: 35 - protein, 112 aa, synthetic construct
  REGION 1..112: note = Synthetic
AIVMTQTSSPVSAVVGGTVAINCQASQSIYNNNWLSWFQQKPGQPPKLLIYLASTLASGVPSRFKGSGSGTQFTLTISDVVCDDAATYYCIGGESSNNDGIAFGGGTEVVVK

SEQ ID NO: 36 - protein, 112 aa, synthetic construct
  REGION 1..112: note = Synthetic
DIVMSQSPSSLAVSAGEKVTMTCKSSQSLLHTSTRKNYLAWYQQKPGQSPKLLIYWASTRESGVPDRFTGSGSGTDFTLTISSVQAEDLAVYYCKQSYDVVTFGAGTKLELK

SEQ ID NO: 37 - protein, 122 aa, synthetic construct
  REGION 1..122: note = Synthetic
QVHLQQSGAELAKPGASVKMSCKASGYTFTSYWIHWIKQRPGQGLEWIGYINPSSGYHEYNQKFIDKATLTADRSSSTAYMHLTSLTSEDSAVYYCARSGWLIHGDYYFDFWGQGTTLTVSS